SEQUENCE LISTING (23 sequences)

SEQ ID NO: 1 - DNA, 507 bp, Ornithodoros moubata
atgctggttttggtgaccctgattttctccttttctgcgaacatcgcatatgctgacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 2 - protein, 168 aa, Ornithodoros moubata
MLVLVTLIFSFSANIAYADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 3 - DNA, 453 bp, Ornithodoros moubata
gacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 4 - protein, 150 aa, Ornithodoros moubata
DSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 5 - DNA, 450 bp, Ornithodoros moubata
agcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 6 - protein, 149 aa, Ornithodoros moubata
SESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 7 - DNA, 447 bp, Ornithodoros moubata
gaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 8 - protein, 148 aa, Ornithodoros moubata
ESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 9 - DNA, 444 bp, Ornithodoros moubata
agcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 10 - protein, 147 aa, Ornithodoros moubata
SDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 11 - DNA, 441 bp, Ornithodoros moubata
gactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 12 - protein, 146 aa, Ornithodoros moubata
DCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 13 - DNA, 438 bp, Ornithodoros moubata
tgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 14 - protein, 145 aa, Ornithodoros moubata
CTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 15 - protein, 20 aa, Artificial Sequence
  PAS sequence
ASPAAPAPASPAAPAPSAPA

SEQ ID NO: 16 - protein, 20 aa, Artificial Sequence
  PAS sequence
AAPASPAPAAPSAPAPAAPS

SEQ ID NO: 17 - protein, 20 aa, Artificial Sequence
  PAS sequence
APSSPSPSAPSSPSPASPSS

SEQ ID NO: 18 - protein, 20 aa, Artificial Sequence
  PAS sequence
SAPSSPSPSAPSSPSPASPS

SEQ ID NO: 19 - protein, 20 aa, Artificial Sequence
  PAS sequence
SSPSAPSPSSPASPSPSSPA

SEQ ID NO: 20 - protein, 24 aa, Artificial Sequence
  PAS sequence
AASPAAPSAPPAAASPAAPSAPPA

SEQ ID NO: 21 - protein, 20 aa, Artificial Sequence
  PAS sequence
ASAAAPAAASAAASAPSAAA

SEQ ID NO: 22 - protein, 751 aa, Artificial Sequence
  PAS-Coversin
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 23 - protein, 150 aa, Artificial Sequence
  Coversin variant 2
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWQSDAGADAVEVECCRQKLEELASGRNQMYPHLKGC

(19) United States
(12) Patent Application Publication
(10) Pub. No.: US 2022/0047673 A1
(43) Pub. Date: Feb. 17, 2022

(54) Coversin for Use in the Treatment of Rheumatic Diseases

(71) Applicants: Volution Immuno Pharmaceuticals SA, 1211 Geneve (CH); The General Hospital Corporation, Boston, MA (US)

(72) Inventors: Miles Nunn, 3 Geneve (CH); Andrew Luster, Wellesley, MA (US); Yoshishige Miyabe, Brookline, MA (US)

(73) Assignees: Volution Immuno Pharmaceuticals SA, 1211 Geneve 3 (CH); The General Hospital Corporation, Boston, MA (US)

(21) Appl. No.: 17/274,751

(22) PCT Filed: Sep. 10, 2019
(86) PCT No.: PCT/EP2019/074101

§ 371 Date: Mar. 9, 2021

Related U.S. Application Data
(60) Provisional application No. 62/729,068, filed on Sep. 10, 2018.

(51) Int. Cl. A61K 38/17; A61K 31/167; A61K 39/395; A61K 38/13; A61K 31/675; A61K 31/4706; A61K 33/242; A61K 31/519; A61K 31/42; A61K 31/5377; A61K 31/655; A61P 19/02

(52) U.S. Cl. CPC A61K 38/1767; A61K 31/167; A61K 39/3955; A61K 38/13; A61K 31/675; A61P 19/02; A61K 33/242; A61K 31/519; A61K 31/42; A61K 31/5377; A61K 31/655; A61K 31/4706

(57) ABSTRACT

The present invention relates to methods of treating or preventing rheumatic disease.

### FIELD OF THE INVENTION

The present invention relates to methods of treating and preventing rheumatic diseases, particularly rheumatoid arthritis.

All documents mentioned in the text and listed at the end of this description are incorporated herein by reference.

### BACKGROUND TO THE INVENTION

### Complement

The complement system is an essential part of the body's natural defence mechanism against foreign invasion and is also involved in the inflammatory process. More than 30 proteins in serum and at the cell surface are involved in the functioning and regulation of the complement system. Recently, it has become apparent that, as well as the approximately 35 known components of the complement system, which may be associated with both beneficial and pathological processes, the complement system itself interacts with at least 85 biological pathways with functions as diverse as angiogenesis, platelet activation and haemostasis, glucose metabolism and spermatogenesis.

The complement system is activated by the presence of materials that are recognised by the immune system as non-self. Three activation pathways exist: (1) the classical pathway which is activated by IgM and IgG complexes or by recognition of carbohydrates; (2) the alternative pathway which is activated by non-self surfaces (lacking specific regulatory molecules) and by bacterial endotoxins; and (3) the lectin pathway which is activated by binding of mannan-binding lectin (MBL) to mannose residues on the surface of a pathogen. The three pathways comprise parallel cascades of events that result in the production of complement activation through the formation of similar C3¹ and C5 convertases on cell surfaces, resulting in the release of acute mediators of inflammation (C3a and C5a) and the formation of the membrane attack complex (MAC). The parallel cascades involved in the classical (here defined as classical via C1q and lectin via MBL) and alternative pathways are shown in FIG. 1. ¹It is conventional to refer to the components of the complement pathway by the letter “C” followed by a number, such as “3”, such that “C3” refers to complement protein C3. Some of these components are cleaved during activation of the complement system and the cleavage products are given lower case letters after the number. Thus, C5 is cleaved into fragments which are conventionally labelled C5a and C5b. The complement proteins do not necessarily act in their number order and so the number does not necessarily give any indication of the order of action. This naming convention is used in this application.

The classical complement pathway, the alternative complement pathway and the lectin complement pathway are herein collectively referred to as the complement pathways. C5b initiates the ‘late’ or ‘terminal’ events of complement activation. These comprise a sequence of polymerization reactions in which the terminal complement components interact to form the MAC, which creates a pore in the cell membranes of some pathogens which can lead to their death or activates the body's own cells without causing lysis. The terminal complement components include C5b (which initiates assembly of the membrane attack system), C6, C7, C8 and C9.

### LTB4

Leukotriene B4 (LTB4) is the most powerful chemotactic and chemokinetic eicosanoid described and promotes adhesion of neutrophils to the vascular endothelium via upregulation of integrins [1]. It is also a complete secretagogue for neutrophils, induces their aggregation and increases microvascular permeability. LTB4 recruits and activates natural killer cells, monocytes and eosinophils. It increases superoxide radical formation [2] and modulates gene expression including production of a number of proinflammatory cytokines and mediators which may augment and prolong tissue inflammation [3,4]. LTB4 also has roles in the induction and management of adaptive immune responses. For example regulation of dendritic cell trafficking to draining lymph nodes [5,6], Th2 cytokine IL-13 production from lung T cells [7], recruitment of antigen-specific effector CD8+ T cells [8] and activation and proliferation of human B lymphocytes [9].

LTB4 and the hydroxyeicosanoids mediate their effects though the BLT1 and BLT2 G-protein coupled receptors [10,11]. Human BLT1 is a high affinity receptor (Kd 0.39-1.5 nM; [12]) specific for LTB4 with only 20-hydroxy LTB4 and 12-epi LTB4 able to displace LTB4 in competitive binding studies [13]. Human BLT2 has a 20-fold lower affinity (Kd 23 nM) for LTB4 than BLT1 and is activated by binding a broader range of eicosanoids including 12-epi LTB4, 20-hydroxy LTB4, 12(S)- and 15(S)-HETE and 12(S)- and 15(S)-HPETE [13]. Human BLT2 has 45.2 and 44.6% amino acid identity with human and mouse BLT1, while human and mouse BLT2 have 92.7% identity [11].

Human BLT1 is mainly expressed on the surface of leukocytes, though it has recently been described in endothelial cells and vascular smooth muscle cells. Human BLT2 is expressed in a broader range of tissue and cell types. A number of specific antagonists of BLT1 and BLT2 have been described which inhibit activation, extravasation and apoptosis of human neutrophils [14] and reduce symptoms caused by neutrophil infiltration in mouse models of inflammatory arthritis [15] and renal ischaemia reperfusion [16]. Increasing numbers of studies indicate that both BLT1 and BLT2 can mediate pathological effects through LTB4 and hydroxyeicosanoids [17], although BLT1 certainly has a dominant role in some pathologies such as collagen induced arthritis in mice [18]. BLT1−/− deficient mice have also highlighted the importance of BLT1 in directing neutrophil migration in inflammatory responses. In particular, a 5LO deficient mouse strain was used to show autocrine activation of BLT1 on neutrophils is needed for their recruitment into arthritic joints [19].

A number of marketed drugs target the eicosanoids. These include the glucocorticoids which modulate phopholipase A2 (PLA2) and thereby inhibit release of the eicosanoid precursor arachidonic acid (AA) [20]. Non-steroidal antiinflammatory drugs (NSAID) and other COX2 inhibitors prevent synthesis of the prostaglandins and thromboxanes [21]. There are also a number of leukotriene (LK) modifiers which either inhibit the 5-LOX enzyme required for LTB4 synthesis and other leukotrienes (Zileuton; [22]), or antagonise the CysLT1 receptor that mediates the effects of cysteinyl leukotrienes (Zafirlukast and Montelukast) [23]. The LK modifiers are orally available and have been approved by the FDA for use in the treatment of e.g. asthma. No drug that acts specifically on LTB4 or its receptors has yet reached the market.

### Rheumatic Diseases

### BACKGROUND

The term rheumatic disease includes conditions causing chronic, often intermittent pain affecting the joints and/or connective tissue. Of the at least 200 different conditions that can be described as rheumatic diseases, rheumatoid arthritis (RA) is a particularly well known example.

Certain rheumatic diseases are caused by injury or infectious diseases, however most are conditions that are caused by autoimmunity. Such conditions include ankylosing spondylitis, relapsing polychondritis, systemic lupus erythematosus, rheumatoid arthritis, gout, inflammatory arthritis, pseudogout, juvenile arthritis, Sjögren syndrome, scleroderma, polymyositis, dermatomyositis, Behçet's disease and psoriatic arthritis.

### Severity

Symptoms of an established arthritis condition include pain and limited function of joints, e.g. joint stiffness, swelling, redness, and warmth. Tenderness of the inflamed joint can be present, as well as fever, fatigue and symptoms from abnormalities of organs such as the lungs, heart, or kidneys caused by the systemic nature of the disease.

Inflammation is thus one key symptom of chronic musculoskeletal diseases, and the resulting joint and muscle pain can be particularly debilitating. Many of these diseases are chronic, progressive, and require long-term medication. An example of a rheumatic disease affecting the joints is arthritis, of which there are many forms. Rheumatoid arthritis (RA) is a particular example, which is associated with inflammation resulting from autoimmunity.

There is an incredible burden on health systems for the treatment of these conditions, because they are highly prevalent chronic diseases with long term effects and no cure. For example, RA affects about 24.5 million people as of 2015 [24], i.e. between 0.5 and 1% of adults in the developed world. In 2013, it resulted in 38,000 deaths up from 28,000 deaths in 1990 [25].

Rheumatoid arthritis (RA) is a chronic, systemic disease characterized by an inflammatory, erosive synovitis. Its pathological diagnosis is hyperplasia of synoviocytes, hyperaemia, thickening of blood vessel walls, infiltration of inflammatory cells, hyperplasia, transparency, and degeneration of fibrotic tissues. Changes in the synovium are marked by the formation of new blood vessels (termed angiogenesis), which play a key role in the formation and maintenance of a pannus of inflammatory vascular tissue. This pannus covers and erodes articular cartilage, eventually leading to joint destruction.

The etiology of RA is not fully understood, but it is considered an autoimmune disorder associated with a pathological angiogenesis and inflammation of affected tissues.

The symptoms that distinguish rheumatoid arthritis from other forms of arthritis are inflammation and soft-tissue swelling of many joints at the same time (polyarthritis). Rheumatoid arthritis is a disabling and painful inflammatory condition, which can lead to substantial loss of mobility due to pain and joint destruction. RA is a systemic disease, often affecting extra-articular tissues throughout the body including the skin, blood vessels, heart, lungs, and muscles.

For rheumatoid arthritis, the basic goal of treatment is to reduce pain and inflammation; to prevent deformation of bone, cartilage, and soft tissues; and to maintain the normal function of the joints; thereby maintaining the normal daily activities of the patients for the longest possible period.

Pharmacological treatment of RA can be divided into disease-modifying antirheumatic drugs (DMARDs), anti-inflammatory agents and analgesics. The goals of treatment are to minimize symptoms such as pain and swelling, to prevent bone deformity (for example, bone erosions visible in X-rays), and to maintain day-to-day functioning.

DMARDs have been found to e.g. improve symptoms, decrease joint damage, and improve overall functional abilities. Antiinflammatories and analgesics improve pain and stiffness but do not prevent joint damage or slow the disease progression.

Examples of DMARDs include traditional small molecules (cyclosporine, cyclophosphamide, hydroxychloroquine, gold salts methotrexate, leflunomide, methotrexate (MTX), mycophenolate and sulfasalazine) and biological agents with immuno-modulatory properties, e.g. produced through genetic engineering such as: tumor necrosis factor alpha (TNFa) blockers (etanercept, certolizumab pegol, golimumab, infliximab, and adalimumab), interleukin-1 blockers (anakinra), anti-B cell (CD20) antibody (rituximab), and blockers of T cell activation (abatacept).

Anti-inflammatory agents for treatment include: non-steroidal antiinflammatory drugs (NSAIDs, most also act as analgesics) and glucocorticoids.

NSAIDs reduce inflammation and pain in the early stage of the disease by inhibiting cyclooxygenase (COX), and therefore the production of prostaglandins (PG), which play a central role in inflammation and pain. NSAIDs can treat symptoms of arthritis, but have little effect on preventing disease progression. Examples include acemetacin, diclofenac, ibuprofen, indomethacin, meloxicam, ketoprofen, sulindac, auranofin, naproxen, nabumetone, piroxicam, mecolfenamic acid, chlofenamic acid, mefenamic acid, pirprofen, fenbufen, tolmetin, flufenamide acid, fenoprofen, methocarbamol and nimesulide. NSAIDs can cause adverse effects resulting from the inhibition of production of COX-I and COX-2 products (e.g. gastrointestinal complex syndrome, upset stomach, abdominal pain, ulcers, gastrointestinal bleeding, as well as damage to the kidneys, liver, and blood system).

Corticosteroids (glucocorticoids such as cortisone and prednisolone) are effective immunosuppresors, but again cannot cure arthritis. Their side effects (including increasing infection, osteoporosis, and dysfunction of the adrenal cortex) increase as the dose and the length of the treatment increase and they are not recommended for use as a long-term medication.

Analgesics are commonly used to treat the pain from arthritis. They include paracetamol, compound analgesics, and opiod analgesics.

Although DMARDs can improve the symptoms of RA, this class of drugs has many serious side effects, and may not be tolerated by patients. Lack of efficacy in some patients, non-tolerability and recurrent secondary infections are factors that have contributed to the need for the development of new therapies. Rheumatoid arthritis treatment remains an unmet clinical need, where approximately 20-40% of rheumatoid arthritis patients do not have an adequate response to any of the currently available therapies.

### Complement Inhibitors

WO 2004/106369 (Evolutec Limited [26]) relates to complement inhibitors. A particular subset of the disclosed complement inhibitors are directed at C5 and prevent C5 being cleaved into C5a and C5b by any of the complement activation pathways. A particular example of such an inhibitor of C5 cleavage is a protein produced by ticks of the species Ornithdoros moubata, which in mature form is a protein consisting of amino acids 19 to 168 of the amino acid sequence shown in FIG. 4 of WO 2004/106369 (SEQ ID NO: 2). The amino acid sequence of the mature form is shown in SEQ ID NO: 4. In WO 2004/106369, this protein is known by the names “EV576” and “OmCI protein” and has more recently been known as “Coversin” [27]. In 2019, Coversin was renamed “nomacopan” following its acceptance as an International Nonproprietary Name by the World Health Organization. This protein is referred to herein as “Coversin”.

In the tick, Coversin is expressed as a pre-protein having a leader sequence comprising amino acids 1 to 18 of the amino acid sequence shown in FIG. 4 of WO 2004/106369 (SEQ ID NO: 2) at the N-terminal end of the mature Coversin protein. The leader sequence is cleaved off after expression. The mature protein has the sequence consisting of amino acids 19 to 168 of the amino acid sequence shown in FIG. 4 of WO 2004/106369 and FIG. 2A of the present application (SEQ ID NO: 2). The amino acid sequence of the mature form is shown in SEQ ID NO: 4.

Coversin also has the ability to inhibit leukotriene B4 (LTB4) activity. The ability to bind LTB4 may be demonstrated by standard in vitro assays known in the art, for example by means of a competitive ELISA between Coversin and an anti-LTB4 antibody competing for binding to labelled LTB4, by isothermal titration calorimetry or by fluorescence titration. There are a number of further patent applications, such as WO 2007/028968, WO 2008/029167, WO 2008/029169, WO 2011/083317 and WO 2016/198133, which relate to the use of Coversin or functional equivalents thereof in various applications. There is no experimental evidence in these applications that confirms the efficacy of Coversin or any functional equivalent thereof in the treatment of rheumatic diseases, such as RA.

In work leading to the present invention, the molecule Coversin which binds LTB4 and which also inhibits the complement pathway by binding to C5, as discussed above, has been shown to both prevent development of arthritis in a mouse model of RA and to ameliorate established arthritic disease in the same model. Coversin has the ability to inhibit both Complement (by inhibiting C5) and also LTB4 and is therefore particularly advantageous in the prevention and treatment of rheumatic diseases, such as RA, either alone or in combination with other treatments. It was previously known that inhibition of either LTB4 or C5a alone can completely prevent development of arthritis in the K/B×N serum transfer mouse model. In the same model inhibition of LTB4 has some ability to ameliorate established arthritic disease. Prior to the present work, it was therefore expected that combined inhibition of C5 activation and LTB4 would be only as effective in the therapeutic model as LTB4 inhibition alone. However, unexpectedly, combined inhibition of C5 and LTB4 by Coversin proved to be much more effective in the therapeutic model than LTB4 inhibition alone.

### SUMMARY OF THE INVENTION

Coversin has been shown to both prevent development of arthritis in a mouse model of RA and to ameliorate established arthritic disease in the same model. Coversin has the ability to inhibit both Complement (by inhibiting C5) and also LTB4 and is therefore particularly advantageous in the prevention and treatment of rheumatic diseases, such as RA, either alone or in combination with other treatments. In Example 1, the administration of Coversin with induction of the disease was shown to completely prevent development of arthritis. By comparison with Zileuton, a greater effect could be observed with the administration of Coversin, as Zileuton only partially ameliorates the development of arthritis in this model. Coversin thus appears to be more effective than an LTB4 inhibitor (Zileuton) in preventing development of arthritis in this mouse model. As noted in the Background to the invention Zileuton inhibits synthesis of LTB4 and also of other LKs some of which may be anti-inflammatory.

It has also been shown (Example 2) that the administration of Coversin after induction of the disease can ameliorate established arthritic disease. The results of this experiment are particularly interesting as they indicate that the dual inhibitory activity of Coversin, targeting both C5 and LTB4 appears to be particularly advantageous in the treatment of arthritis.

In these experiments, a modified Coversin polypeptide, which has reduced or absent C5-binding activity but which retains LTB4-binding ability, was compared to Coversin. This molecule (referred to as L-Coversin) was more effective than Zileuton but less effective than Coversin in ameliorating established arthritic disease, emphasising the advantage of Coversin in the treatment of rheumatic diseases, such as RA.

The present inventors have therefore demonstrated that administration of the tick protein Coversin (also referred to as EV576 and OmCI in the art and herein [26]) can be used to treat or prevent rheumatic diseases.

The invention therefore provides a method of treating or preventing a rheumatic disease, which comprises administering a therapeutically or prophylactically effective amount of an agent which is a protein comprising amino acids 19 to 168 of the amino acid sequence in FIG. 2 (SEQ ID NO: 2) or a functional equivalent of this protein.

The invention also provides an agent which is a protein comprising amino acids 19 to 168 of the amino acid sequence in FIG. 2 (SEQ ID NO: 2) or a functional equivalent of this protein for use in a method of treating or preventing a rheumatic disease.

The invention also provides a method of treating or preventing a rheumatic disease, comprising administering a therapeutically or prophylactically effective amount of an agent which is a nucleic acid molecule encoding a protein comprising amino acids 19 to 168 of the amino acid sequence in FIG. 2 (SEQ ID NO: 2) or a functional equivalent of this protein.

The invention also provides an agent which is a nucleic acid molecule encoding a protein comprising amino acids 19 to 168 of the amino acid sequence in FIG. 2 (SEQ ID NO: 2) or a functional equivalent of this protein for use in a method of treating or preventing a rheumatic disease.

The invention also provides a method of treating or preventing a rheumatic disease, which comprises administering (a) a therapeutically or prophylactically effective amount of an agent which is a protein comprising amino acids 19 to 168 of the amino acid sequence in FIG. 2 (SEQ ID NO: 2) or a functional equivalent of this protein and (b) a second rheumatic disease treatment.

The invention also provides (a) an agent which is a protein comprising amino acids 19 to 168 of the amino acid sequence in FIG. 2 (SEQ ID NO: 2) or a functional equivalent of this protein and (b) a second rheumatic disease treatment, for use in a method of treating or preventing an rheumatic disease.

The invention also provides a method of treating or preventing a rheumatic disease, comprising administering (a) a therapeutically or prophylactically effective amount of an agent which is a nucleic acid molecule encoding a protein comprising amino acids 19 to 168 of the amino acid sequence in FIG. 2 (SEQ ID NO: 2) or a functional equivalent of this protein and (b) a second rheumatic disease treatment.

The invention also provides (a) an agent which is a nucleic acid molecule encoding a protein comprising amino acids 19 to 168 of the amino acid sequence in FIG. 2 (SEQ ID NO: 2) or a functional equivalent of this protein and (b) a second rheumatic disease treatment for use in a method of treating or preventing a rheumatic disease.

The invention also provides a method of reducing the amount of a second rheumatic disease treatment that is required to treat or prevent a rheumatic disease, or reducing the duration of treatment with a second rheumatic disease treatment that is required to treat or prevent a rheumatic disease, said method comprising administering a therapeutically or prophylactically effective amount of an agent which is a protein comprising amino acids 19 to 168 of the amino acid sequence in FIG. 2 (SEQ ID NO: 2) or a functional equivalent of this protein, or a nucleic acid molecule encoding said agent, and said second rheumatic disease treatment.

### DETAILED DESCRIPTION

### Diseases

The subject may have, be suspected of having, or may be at risk of developing a rheumatic disease. In one embodiment the rheumatic disease is arthritis, including subtypes thereof. In one embodiment the rheumatic disease is an autoimmune rheumatic disease. Examples of autoimmune rheumatic diseases include ankylosing spondylitis, relapsing polychondritis, systemic lupus erythematosus, rheumatoid arthritis, gout, inflammatory arthritis, pseudogout, juvenile arthritis, Sjögren syndrome, scleroderma, Polymyositis, Dermatomyositis, Behçet's disease and psoriatic arthritis. In certain embodiments the rheumatic disease is RA. In other embodiments the RA is associated with vasculitis.

Subtypes of arthritis include osteoarthritis, gout (or gouty arthritis), RA, psoriatic arthritis, juvenile rheumatoid arthritis, juvenile idiopathic arthritis and polymyalgia rheumatic. A list of types of arthritis according to the Arthritis Foundation https://www.arthritis.org/about-arthritis/types/is shown below:

TABLE 1
Adult-onset Still’s disease
Ankylosing Spondylitis
Back Pain
Behçet’s Disease
Bursitis
Calcium Pyrophosphate Deposition Disease (CPPD)
Carpal Tunnel Syndrome
Chondromalacia Patella
Chronic Fatigue Syndrome
Complex Regional Pain Syndrome
Cryopyrin-Associated Periodic Syndromes (CAPS)
Degenerative Disc Disease
Developmental-Dysplasia of Hip
Diffuse Idiopathic Skeletal Hyperostosis
Ehlers-Danlos
Familial Mediterranean Fever
Fibromyalgia
Fifth Disease
Giant Cell Arteritis
Gout
Granulomatosis with Polyangiitis
Hemochromatosis
Infectious Arthritis
Inflammatory Arthritis
Inflammatory Bowel Disease
Juvenile Arthritis
Juvenile Dermatomyositis (JD)
Juvenile Idiopathic Arthritis (JIA)
Juvenile Scleroderma
Kawasaki Disease
Lupus
Lupus in Children & Teens
Lyme Disease
Mixed Connective Tissue Disease
Myositis (inc. Polymyositis, Dermatomyositis)
Osteoarthritis
Osteoporosis
Pagets
Palindromic Rheumatism
Patellofemoral Pain Syndrome
Pediatric Rheumatic Diseases
Pediatric SLE
Polymyalgia Rheumatica
Pseudogout
Psoriatic Arthritis
Raynaud’s Phenomenon
Reactive Arthritis
Reflex Sympathetic Dystrophy
Reiter’s Sydrome
Rheumatic Fever
Rheumatism
Rheumatoid Arthritis
Scleroderma
Sjögren’s Disease
Spinal Stenosis
Spondyloarthritis
Systemic Juvenile Idiopathic Arthritis
Systemic Lupus Erythematosus
Systemic Lupus Erythematosus in Children & Teens
Systemic Sclerosis
Temporal Arteritis
Tendinitis
Vasculitis

RA is a preferred disease.

The presence of these diseases may be determined by routine diagnosis that is well understood in the art. The severity of certain conditions can also be scored, which is useful in assessing whether a certain treatment is effective.

For example, for RA clinical scoring may be conducted using clinically accepted criteria such as those established by American College of Rheumatology (ACR). One example is the ACR score which allows the measurement of the amount of improvement after treatment (https://www.rheumatoidarthritis.org/treatment/acr-score/). ACR scores take into account a variety of factors to create a score assessing the amount of improvement a patient's rheumatoid arthritis has made. ACR criteria also assess and establish improvement in tender and painful joint counts, as well as improvement in three of five of the following parameters:
(1) Inflammation—Laboratory tests, either measuring the erythrocyte sedimentation rate (ESR) or C-reactive proteins (CRPs), determine the amount of inflammation in the joints.
(2) Patient assessment—The patient describes and determines their own assessment of their progress.
(3) Physician assessment—The physician describes and determines the patient's progress.
(4) Pain scale—How much the pain the patient feels in his or her joints on a daily base.
(5) Disability/functionality questionnaire—How much the patient is able to use his or her joints easily to complete daily activities.

A score of ACR 20, means that that patient has improved their tender and painful joint counts by 20 percent, as well as made a 20 percent improvement in three of the five above parameters. ACR20 measures a 20% improvement on a scale of 28 intervals. ACR50 and ACR70 correspond to 50% and 70% improvements.

An alternative is the Disease Activity Score 28-joint count (DAS28). In one embodiment a DAS28 score >5.1 is considered as severe, 3.2 to 5.1 is considered moderate and a patient scoring less than 2.6 is defined as being in remission. Further, a decrease in DAS28 score of 0.6 or less after a treatment is considered to show a poor response, while decreases greater than 1.2 points indicate a moderate or good response, dependent on whether an individual's DAS28 score at the end point is above or below 3.2 respectively.

A further alternative is Clinical Disease Activity Index (CDAI). A CDAI score >10 to <22 is considered moderate and a CDAI score >22 is considered as severe, CDAI>2.8 and <10 is considered low disease activity and CDAI<2.8 is considered to be remission. A CDAI reduction of 6.5 represents moderate improvement. In a further alternative embodiment a RAPID3 [28] score of from 1 to 3 indicates near remission, a score from 4 to 6 indicates low severity, a score from 7 to 12 is considered moderate and a RAPID3 score of 13 to 30 is considered as severe.

Subjects at risk of developing a rheumatic disease may benefit from administration of the agents referred to herein, in order to prevent the rheumatic disease or symptoms thereof. Risk factors for certain rheumatic diseases are set out in Table 2 below and are discussed in more detail in [29]:

TABLE 2

| | Host/Environmental risk | |
|---|---|---|
| Disease | factor | Gene |
| Rheumatoid arthritis | Diet (caffeine and | PTPN22 |
| | Mediterranean diet) | |
| | Smoking | PADI4 |
| | Hormones | CTLA4 |
| | | FCRL3 |
| | | MHC2A |
| | | HLA DRB1 |
| Juvenile idiopathic arthritis | | Macrophage inhibitory factor |
| | | (MIF) |
| | | PTPN22 |
| | | NRAMP1 |
| | | IL-6 |
| Psoriatic arthritis | Rubella vaccination | CARD15 |
| | Injury requiring medical | MICA |
| | consultation | |
| | Recurrent oral ulcers | TNF |
| | Moving house | IL |
| | Corticosteroids | |
| | Pregnancy | |
| Ankylosing spondylitis | | ARTS1 |
| | | 1L-23R |
| | | IL-1 gene cluster |
| | | Cytochrome P450 2D6 |
| | | (CYP2D6) gene |
| Systemic lupus erythematosus | Breast-feeding | MHC |
| | Early natural menopause | ITGAM |
| | Lipstick | IRF5 |
| | | BLK |
| | | STA T4 |
| | | PTPN22 |
| | | FCGR2A |
| Scleroderma | Exposure to silica or organic | Familial risk |
| | solvents | |
| | | HLA-DQA1 |
| | | Fibrillin-1 SNP haplotypes |
| | | TGF-β |
| | | CTGF |
| | | Foetal microchimerism |
| Osteoarthritis | Obesity/Body mass index | IL-1 gene cluster |
| | Physical activity | Frizzled-related protein-3 |
| | | (FRZB) gene |
| | Grip strength | Matrilin-3 gene |
| | Previous injury | IL-4 receptor |
| | | Metalloproteinase gene |
| | | ADAM12 |
| | | Asporin (ASPN) gene |
| | | Estrogen receptor |
| Gout | High purine diet | TNF-α promoter |
| | Dairy products | |
| | Hypertension | |
| | Pharmacologic agents | |
| Fibromyalgia and chronic | Physical trauma | Serotonin transporter gene |
| widespread pain | | |
| | Somatisation | Familial risk |
| | Health-seeking behaviour | COMT |
| | Poor sleep | |

Subjects having one or more of these risk factors are preferred, in terms of treatment or prevention of rheumatic disease.

In some embodiments a subject may have one or more of these risk factors but may not show clinical symptoms. In some embodiments the subject may be in the effector phase of a condition, e.g. RA. In such subjects one or more autoantibody associated with RA may be detected in said subject, e.g. at levels indicative of a risk of disease progression. The K/B×N mouse model of RA is a model of the effector phase of RA, and in this phase there is autoantibody involvement. Without being bound by theory Coversin may be particularly effective in treatment where autoantibody is present or wherein the rheumatic disease continues to be driven by autoantibodies. In certain embodiments any method of treatment may further comprise the step of selecting a subject for treatment on the basis of the presence of autoantibodies.

### RA

In certain embodiments the rheumatic disease is RA. The RA subject may be seropositive or seronegative (according to the presence or absence of detectable rheumatoid factor (RF), an antibody directed to the Fc portion of human IgG, which can be determined e.g. by agglutination reactions or nephelometry). Other antibodies with specificities for RA are also known, including antibodies to certain peptides containing citrullinated arginine residues (anti-citrullinated protein antibodies (ACPAs)). The RA subject may additionally or alternatively have autoantibodies to cyclic citrullinated peptides (CCP) (which can be detected using commercially available tests). ACPAs allow the diagnosis of rheumatoid arthritis (RA) to be made at a very early stage and ACPA testing forms part of the 2010 ACR-EULAR classification criteria for rheumatoid arthritis.

The RA may therefore be described as autoantibody driven RA, and in particular the subjects may be RF positive and/or anti-CCP (or ACPA) positive. In certain embodiments any method of treatment may further comprise the step of selecting a subject for treatment on the basis of the presence of RF and/or anti-CCP autoantibodies (or ACPA).

### Vasculitis

In a further embodiment the RA subject may have vasculitis associated with RA, e.g. rheumatoid vasculitis, e.g. systemic vasculitis. The term ‘vasculitis’ indicates that blood vessels are inflamed. The consequences of vasculitis depend on the size, site and number of blood vessels involved. Infarction of the tissue that the blood vessel supplies may occur when small or medium-sized arteries are involved (e.g. coronary artery vasculitis can result in a heart attack) but the effects are less serious when very small blood vessels such as capillaries are involved. An exception is when there is extensive local vasculitis, such as can occur in the kidney, resulting in glomerulonephritis.

Vasculitis can occur as a complication of rheumatoid arthritis, e.g. small vessel vasculitis (e.g. involving small arteries and arterioles). Vasculitis is also associated with most of the extra-articular (meaning ‘outside of the joints’) manifestations described in rheumatoid arthritis. These include inflammation of the eyes (iritis), inflammation of the lining of the heart and lung (pericarditis and pleurisy) and other lung and heart manifestations including inflammation of the bases of the lung (fibrosing alveolitis) and irregular heartbeat, including heart block. Systemic vasculitis subjects usually have high levels of RF in their blood

Systemic vasculitis is a rare but serious complication of rheumatoid arthritis and may be considered one of the most serious extra-articular consequences of this disease. By way of example rheumatoid vasculitis (RV) which is characterised by inflammation of mid-sized arteries and capillaries is severe and up to 40% of patients die within 5 years due to damage from vasculitis and/or consequences of immunosuppressive therapy. RV remains difficult to treat and is associated with a high mortality.

The subject may have previously been treated with one or more other rheumatic disease treatments (e.g. one or more of the treatments or DMARDs discussed above). In some embodiments the previous treatment has not been effective, has ceased to be effective, or was discontinued owing to one or more adverse events. Discontinuing one rheumatic disease treatment is relatively common, particularly for TNF inhibitors. In certain embodiments therefore the subject has previously been treated with one or more TNF inhibitors. In certain embodiments such treatment was discontinued owing to lack of efficacy or adverse effects. A lack of efficacy may be assessed as discussed elsewhere herein. Examples of adverse effects include coughing, headaches, heartburn, nausea or vomiting, stomach pain, weakness, serious allergic reaction, infection, development of cancer, e.g. a tumour.

### Timing

It can be advantageous to start treatment early after diagnosis or after disease onset. In a preferred embodiment of the invention the treatment of rheumatic diseases in subjects according to the invention starts not more than about 6 months from first diagnosis, not more than about 12 months from first diagnosis, not more than about 18 months from first diagnosis, not more than about 2 years from first diagnosis, not more than about 3 years from first diagnosis, not more than about 4 years from first diagnosis, not more than about 5 years from first diagnosis.

In a preferred embodiment of the invention the treatment of rheumatic diseases in subjects according to the invention is in subjects having not more than about 6 months disease duration, not more than about 12 months disease duration, not more than about 18 months disease duration, not more than about 2 years disease duration, not more than about 3 years disease duration, not more than about 4 years disease duration, not more than about 5 years disease duration.

Typically the treatment of rheumatic disease is in subjects during an acute phase of the disease. The term “acute phase” in the context of rheumatoid arthritis shall be taken to mean a patient experiencing significant inflammation of one or more joints, as opposed to only mild or moderate inflammation. The acute phase is also referred to as “flares” in the art.

### Outcomes of Administration

The subject may, as a result of the treatment, have reduced incidence of symptoms, alleviation of symptoms, inhibition or delay of occurrence or re-occurrence of symptoms, or a combination thereof. Preferably the treatment gives rise to a reduction in the typical disease condition symptoms. For example, this may be manifest in reducing the amount of inflammation or swelling, in the joint size, number of joints affected, or in the amount of pain or longer interval between relapses. There may also be a reduction in fever and/or in the severity and/or number of sores. A proportion of subjects may have complete resolution of symptoms and may have no further relapses.

As a result of the treatment the subject may exhibit an improvement in their clinical score, e.g. using one of the methods referred to above. For example, a subject may be considered in remission after treatment or may have a score after a certain period of treatment that indicates an improvement in symptoms. Where the subject has a rheumatic disease which is scored by DAS28, CDAI or RAPID3, e.g. RA, treatment may in one embodiment give rise to an improvement in DAS28 score, CDAI or RAPID3. A subject may have a decrease in DAS28 of at least 0.8 or 1.0 or 1.2 points (preferably where the subject's DAS28 score at the end point is below 3.2) after a course of treatment. A subject may have a CDAI reduction of at least 6.5 after a course of treatment. In a further alternative embodiment a subject may have a RAPID3 score of less than 6 after a course of treatment.

Alternatively stated, a subject may have a score of ACR20, ACR50 or ACR70 after a course of treatment.

Methods for diagnosing acute rheumatoid arthritis are known in the art and may include the analysis of erythrocyte sedimentation rate (ESR) and/or serum C-reactive protein (CRP) levels wherein e.g. ESR of 50 or above is considered to be acute. Where the subject has acute RA the treatment may result in a reduction in erythrocyte sedimentation rate (ESR) and/or serum C-reactive protein (CRP) levels.

Where the subject has RV the treatment may result in an improvement as described above and/or an improvement in the clinical manifestations of RV (which include cutaneous ulcers, peripheral gangrene, vascular neuropathy, inflammatory eye disease and visceral infarction, i.e. there may be a reduction in one or more of these clinical manifestations and/or a reduction in the severity thereof).

The treatment may also result in a reduction in the amount or duration of a second rheumatic disease treatment that is required.

Thus in a further embodiment of the invention, there is provided a method of reducing the clinical score in a subject with a rheumatic disease, or reducing joint inflammation in a subject with rheumatic disease, or reducing joint stiffness in a subject with rheumatic disease, said method comprising administering a therapeutically or prophylactically effective amount of an agent which is a protein comprising amino acids 19 to 168 of the amino acid sequence in FIG. 2 (SEQ ID NO: 2) or a functional equivalent of this protein, or a nucleic acid molecule encoding said agent. This may be alone or with a second rheumatic disease treatment.

The invention also provides an agent which is a protein comprising amino acids 19 to 168 of the amino acid sequence in FIG. 2 (SEQ ID NO: 2) or a functional equivalent of this protein, a nucleic acid molecule encoding said agent, for use in a method of reducing the the clinical score in a subject with a rheumatic disease, or reducing joint inflammation in a subject with rheumatic disease, or reducing joint stiffness in a subject with rheumatic disease. This may be alone or with a second rheumatic disease treatment.

The agent of the invention can be used in combination with other rheumatic disease treatments, as discussed above. The combination of the agent of the invention with the other (referred to here as a “second”) rheumatic disease treatment may be such that the amount of the second rheumatic disease agent is reduced in comparison to the amount that is used in the absence of treatment with the agent of the invention, or the duration of the treatment with second rheumatic disease agent is reduced in comparison to the duration of treatment that is used in the absence of treatment with the agent of the invention. This is advantageous in view of the side effects of certain known treatments. Therefore, there is also provided a method of reducing the amount of a second rheumatic disease treatment that is used for the treatment or reducing the duration of the treatment with a second rheumatic disease treatment.

Preferably the second rheumatic disease treatment is selected from a DMARD (e.g. one or more of cyclosporine, cyclophosphamide, hydroxychloroquine, gold salts, methotrexate, leflunomide, mycophenolate, sulfasalazine, etanercept, certolizumab pegol, golimumab, infliximab, and adalimumab, anakinra, rituximab and abatacept), an anti-inflammatory agent (e.g. an NSAID, or a glucocorticoid) and an analgesic (e.g. selected from paracetamol, compound analgesics and opiod analgesics).

When the agent of the invention and a second rheumatic disease treatment are used, they may be administered together or separately. The agent of the invention may be administered first and the second rheumatic disease treatment may be administered second, or vice versa.

Thus, where the agent of the invention is used in combination with one or more other rheumatic disease treatments, e.g. in methods described as above, this can be described an agent which is a protein comprising amino acids 19 to 168 of the amino acid sequence in FIG. 2 (SEQ ID NO: 2) or a functional equivalent of this protein for use in a method of treating or preventing rheumatic disease with a second rheumatic disease treatment, or as a second rheumatic disease treatment for use in a method of treating or preventing rheumatic disease with an agent which is a protein comprising amino acids 19 to 168 of the amino acid sequence in FIG. 2 (SEQ ID NO: 2) or a functional equivalent of this protein.

Where the treatment gives rise to a reduction in the amount or duration of the second rheumatic disease treatment, the reduction may be up to or at least 10, 20, 30, 40, 50, 60, 70, 80% compared to the amount of the second treatment that is used in the absence of the agent of the invention.

### Subjects

Preferred subjects, agents, doses and the like are as disclosed herein.

Any reference to any reduction or increase is a reduction or increase in a disease parameter is compared to said subject in the absence of the treatment. Preferably, the parameter can be quantitated and where this is the case the increase or decrease is preferably statistically significant. For example the increase or decrease may be at least 3, 5, 10, 15, 20, 30, 40, 50% or more compared to the parameter in the absence of treatment (e.g. before said treatment is started).

The subject to which the agent is administered in the practice of the invention is preferably a mammal, preferably a human. The subject to which the agent is administered is at risk of a rheumatic disease or a subject who has a rheumatic disease.

Methods of the invention may also comprise one or more additional steps of (i) determining whether the subject is at risk of or has rheumatic disease, (ii) determining the severity of the rheumatic disease, which may be carried out before and/or after administration of Coversin.

### Agent to be Used in the Invention

According to one embodiment of the invention, the agent is Coversin itself or a functional equivalent thereof. In the following, the term “a Coversin-type protein” is used as shorthand for “a protein comprising amino acids 19 to 168 of the amino acid sequence shown in FIG. 2 (SEQ ID NO: 2) or a functional equivalent thereof”.

Coversin was isolated from the salivary glands of the tick Ornithodoros moubata. Coversin is an outlying member of the lipocalin family and is the first lipocalin family member shown to inhibit complement activation. Coversin inhibits the classical, alternative and lectin complement pathways by binding to C5 and preventing its cleavage by C5 convertase into C5a and C5b, thus inhibiting both the production of C5a, which is an active (e.g. proinflammatory) peptide, and the formation of the MAC. Coversin has been demonstrated to bind to C5 and prevent its cleavage by C5 convertase in rat, mouse and human serum with an IC50 of approximately 0.02 mg/ml.

A Coversin-type protein may thus comprise or consist of amino acids 19 to 168 of the amino acid sequence in FIG. 2 (SEQ ID NO: 2) or amino acids 1 to 168 of the amino acid sequence in FIG. 2 (SEQ ID NO: 2). The first 18 amino acids of the protein sequence given in FIG. 2 form a signal sequence which is not required for C5 binding or for LTB4 binding activity and so this may optionally be dispensed with, for example, for efficiency of recombinant protein production.

The Coversin protein has been demonstrated to bind to C5 with a Kd of 1 nM, determined using surface plasmon resonance (SPR) [30]. Coversin-type peptides (e.g. functional equivalents of the Coversin protein) preferably retain the ability to bind C5, conveniently with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [30].

Coversin inhibits the classical complement pathway, the alternative complement pathway and the lectin complement pathway. Preferably, a Coversin-type protein binds to C5 in such a way as to stabilize the global conformation of C5 but not directly block the C5 cleavage site targeted by the C5 convertases of the three activation pathways. Binding of Coversin to C5 results in stabilization of the global conformation of C5 but does not block the convertase cleavage site. Functional equivalents of Coversin also preferably share these properties.

C5 is cleaved by the C5 convertase enzyme (FIG. 1). The products of this cleavage include an anaphylatoxin C5a and a lytic complex C5b which promotes the formation of a complex of C5b, C6, C7, C8 and C9, also known as membrane attack complex (MAC). C5a is a highly pro-inflammatory peptide implicated in many pathological inflammatory processes including neutrophil and eosinophil chemotaxis, neutrophil activation, increased capillary permeability and inhibition of neutrophil apoptosis [31].

Monoclonal antibodies and small molecules that bind and inhibit C5 have been developed to treat various diseases [32], in particular PNH, psoriasis, rheumatoid arthritis, systemic lupus erythematosus and transplant rejection. However, some of these monoclonal antibodies do not bind to certain C5 proteins from subjects with C5 polymorphisms, and are thus ineffective in these subjects [33]. Preferably, the Coversin-type protein binds to and inhibits cleavage of not only wild-type C5 but also C5 from subjects with C5 polymorphisms (e.g. C5 polymorphisms that render treatment by eculizumab ineffective, or reduce the efficacy of treatment with eculizumab). The term “C5 polymorphism” includes any version of C5 which has been changed by insertion, deletion, amino acid substitution, a frame-shift, truncation, any of which may be single or multiple, or a combination of one or more of these changes compared to the wild-type C5. In a human subject, wild-type C5 is considered the C5 protein with accession number NP_001726.2; version GI:38016947. Examples of C5 polymorphisms include polymorphisms at amino acid position 885, e.g. Arg885Cys (encoded by c.2653C>T) p.Arg885His (encoded by c.2654G>A) and Arg885Ser, which decrease the effectiveness of the mAb eculizumab [33].

The ability of an agent to bind C5, including C5 from subjects with C5 polymorphisms, e.g. C5 polymorphisms that render treatment by eculizumab ineffective, or reduce the efficacy of treatment with eculizumab may be determined by standard in vitro assays known in the art, for example by surface plasmon resonance or western blotting following incubation of the protein on the gel with labelled C5. Preferably, the Coversin-type protein binds C5, either wild-type and/or C5 from subjects with C5 polymorphisms, e.g. C5 polymorphisms that render treatment by eculizumab ineffective, or reduce the efficacy of treatment with eculizumab, with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [30].

It may show higher, lower or the same affinity for wild-type C5 and C5 from subjects with C5 polymorphisms, e.g. C5 polymorphisms that render treatment by eculizumab ineffective, or reduce the efficacy of treatment with eculizumab.

The ability of a Coversin-type protein to inhibit complement activation may also be determined by measuring the ability of the agent to inhibit complement activation in serum. For example, complement activity in the serum can be measured by any means known in the art or described herein.

The Coversin-type protein may also be defined as having the function of inhibiting eicosanoid activity. Coversin has also been demonstrated to bind LTB4. Functional equivalents of the Coversin protein may also retain the ability to bind LTB4 with a similar affinity as the Coversin protein.

The ability of a Coversin-type protein to bind LTB4 may be determined by standard in vitro assays known in the art, for example by means of a competitive ELISA between Coversin and anti-LTB4 antibody competing for binding to labelled LTB4, by isothermal titration calorimetry or by fluorescence titration. Data obtained using fluorescence titration shows that Coversin binds to LTB4 with a Kd of between 200 and 300 pM. For example, binding activity for LTB4 (Caymen Chemicals, Ann Arbor, Mich., USA) in phosphate buffered saline (PBS) can be quantified in a spectrofluorimeter e.g. a LS 50 B spectrofluorimeter (Perkin-Elmer, Norwalk, Conn., USA). This may be carried out by may be carried out as follows:

Purified 100 nM solutions of Coversin, in 2 mL PBS were applied in a quartz cuvette (10 mm path length; Hellma, Mühlheim, Germany) equipped with a magnetic stirrer. Temperature was adjusted to 20° C. and, after equilibrium was reached, protein Tyr/Trp fluorescence was excited at 280 nm (slit width: 15 nm). The fluorescence emission was measured at 340 nm (slit width: 16 nm) corresponding to the emission maximum. A ligand solution of 30 μM LTB4 in PBS was added step-wise, up to a maximal volume of 20 μL (1% of the whole sample volume), and after 30 s incubation steady state fluorescence was measured. For calculation of the KD value, data was normalized to an initial fluorescence intensity of 100%, the inner filter effect was corrected using a titration of 3 μM N-acetyl-tryptophanamide solution and data was plotted against the corresponding ligand concentration. Then, non-linear least squares regression based on the law of mass action for bimolecular complex formation was used to fit the data with Origin software version 8.5 (OriginLab, Northampton, Mass., USA) using a published formula (Breustedt et al., 2006) [34].

Coversin may bind LTB4 with an with a Kd of less than 1 nM, more conveniently less than 0.9 nM, most conveniently less than 0.8 nM, preferably less than 0.7 nM, more preferably less than 0.6 nM, most preferably less than 0.5 nM, even more preferably less than 0.4 nM, and advantageously less than 0.3 nM, wherein said Kd is determined using fluorescence titration, preferably in accordance with the method above. The Coversin-type protein preferably shares these properties.

According to one embodiment of the invention, the Coversin-type protein may bind to both C5 and to LTB4, e.g. to both wild-type C5 and C5 from subjects with C5 polymorphisms, e.g. C5 polymorphisms that render treatment by eculizumab ineffective, or reduce the efficacy of treatment with eculizumab, and to LTB4.

The Coversin-type protein may thus act to prevent the cleavage of complement C5 by C5 convertase into complement C5a and complement C5b, and also to inhibit LTB4 activity. Using an agent which binds to both C5 and LTB4 is particularly advantageous. C5 and the eicosanoid pathway may both contribute to the observed pathology in rheumatic diseases. Thus by using a single agent which inhibits multiple pathways involved in the rheumatic diseases an enhanced effect can be achieved, compared to using an agent which inhibits only a single pathway involved in the inflammatory effects of complement-mediated diseases and disorders (see e.g. example 2 comparing Coversin and L-Coversin). There are furthermore practical advantages associated with administering a single molecule.

Preferably, the agent of the invention is derived from a haematophagous arthropod. The term “haematophagous arthropod” includes all arthropods that take a blood meal from a suitable host, such as insects, ticks, lice, fleas and mites. Preferably, the agent is derived from a tick, preferably from the tick Ornithodoros moubata.

A functional equivalent of Coversin may be a homologue or fragment of Coversin which retains its ability to bind to C5, either wild-type C5 or C5 from a subject with a C5 polymorphism (e.g. the Arg885Cys, Arg885His or Arg885Ser polymorphisms described above), and to prevent the cleavage of C5 by C5 convertase into C5a and C5b. The homologue or fragment may also retain its ability to bind LTB4.

Homologues include paralogues and orthologues of the Coversin sequence that is explicitly identified in FIG. 2 (SEQ ID NO: 2), including, for example, the Coversin protein sequence from other tick species, including Rhipicephalus appendiculatus, R. sanguineus, R. bursa, A. americanum, A. cajennense, A. hebraeum, Boophilus microplus, B. annulatus, B. decoloratus, Dermacentor reticulatus, D. andersoni, D. marginatus, D. variabilis, Haemaphysalis inermis, Ha. leachii, Ha. punctata, Hyalomma anatolicum anatolicum, Hy. dromedarii, Hy. marginatum marginatum, Ixodes ricinus, I. persulcatus, I. scapularis, I. hexagonus, Argas persicus, A. reflexus, Ornithodoros erraticus, O. moubata moubata, O. m. porcinus, and O. savignyi.

The term “homologue” is also meant to include the equivalent Coversin protein sequence from mosquito species, including those of the Culex, Anopheles and Aedes genera, particularly Culex quinquefasciatus, Aedes aegypti and Anopheles gambiae; flea species, such as Ctenocephalides felis (the cat flea); horseflies; sandflies; blackflies; tsetse flies; lice; mites; leeches; and flatworms. The native Coversin protein is thought to exist in O. moubata in another three forms of around 18 kDa and the term “homologue” is meant to include these alternative forms of Coversin.

Methods for the identification of homologues of the Coversin sequence given in FIG. 2 will be clear to those of skill in the art. For example, homologues may be identified by homology searching of sequence databases, both public and private. Conveniently, publicly available databases may be used, although private or commercially-available databases will be equally useful, particularly if they contain data not represented in the public databases. Primary databases are the sites of primary nucleotide or amino acid sequence data deposit and may be publicly or commercially available. Examples of publicly-available primary databases include the GenBank database (http://www.ncbi.nlm.nih.gov/), the EMBL database (http://www.ebi.ac.uk/), the DDBJ database (http://www.ddbj.nig.acjp/), the SWISS-PROT protein database (http://expasy.hcuge.ch/), PIR (http://pir.georgetown.edu/), TrEMBL (http://www.ebi.ac.uk/), the TIGR databases (see http://www.tigr.org/tdb/index.html), the NRL-3D database (http://www.nbrfa.georgetown.edu), the Protein Data Base (http://wwwscsb.org/pdb), the NRDB database (ftp://ncbi.nlm.nih.gov/pub/nrdb/README), the OWL database (http://www.biochem.ucl.ac.uk/bsm/dbbrowser/OWL/) and the secondary databases PROSITE (http://expasy.hcuge.ch/sprot/prosite.html), PRINTS (http://iupab.leeds.ac.uk/bmb5dp/prints.html), Profiles (http://ulrec3.unil.ch/software/PFSCAN_form.html), Pfam (http://www.sanger.ac.uk/software/pfam), Identify (http://dna.stanford.edu/identify/) and Blocks (http://www.blocks.fhcrc.org) databases. Examples of commercially-available databases or private databases include PathoGenome (Genome Therapeutics Inc.) and PathoSeq (previously of Incyte Pharmaceuticals Inc.).

Typically, greater than 30% identity between two polypeptides (preferably, over a specified region such as the active site) is considered to be an indication of functional equivalence and thus an indication that two proteins are homologous. Preferably, proteins that are homologues have a degree of sequence identity with the Coversin protein sequence identified in FIG. 2 (SEQ ID NO: 2) of greater than 60%. More preferred homologues have degrees of identity of greater than 70%, 80%, 90%, 95%, 98% or 99%, respectively with the Coversin protein sequence given in FIG. 2 (SEQ ID NO:2). Homologues may have degrees of identity of greater than 70%, greater than 80%, greater than 90%, greater than 95%, greater than 98% or greater than 99%, respectively, with the Coversin protein sequence given in FIG. 2 (SEQ ID NO: 2). In some embodiments, homologues have a degree of identity of greater than 90% with the Coversin protein sequence given in FIG. 2 (SEQ ID NO: 2). Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1]. The % identity may be over the full length of the relevant reference sequence (e.g. amino acids 1-168 of SEQ ID NO:2 or amino acids 19-168 of SEQ ID NO:2).

Coversin-type proteins thus can be described by reference to a certain % amino acid sequence identity to a reference sequence e.g. amino acids 19-168 of FIG. 2, SEQ ID NO:2 or amino acids 1-168 of FIG. 2, SEQ ID NO:2 e.g. as a protein comprising or consisting of a sequence having at least 60%, 70%, 80%, 90%, 95%, 98% or 99% identity to amino acids 19-168 of FIG. 2, SEQ ID NO:2 or amino acids 1-168 of FIG. 2, SEQ ID NO:2). In some embodiments, Coversin-type proteins comprise or consist of a sequence having at least 90% identity with the Coversin protein sequence of SEQ ID NO: 4. Where the Coversin-type protein comprises said sequence, the Coversin-type protein may be a fusion protein (with e.g. a second protein, e.g. a heterologous protein). Suitable second proteins are discussed below.

In the various aspects and embodiments of this disclosure, the modified Coversin polypeptides (e.g. Coversin-type proteins) may differ from the unmodified Coversin polypeptides in SEQ ID NO: 2 and SEQ ID NO: 4 by from 1 to 50, 2-45, 3-40, 4-35, 5-30, 6-25, 7-20, 8-25, 9-20, 10-15 amino acids, up to 1, 2, 3, 4, 5, 7, 8, 9, 10, 20, 30, 40, 50 amino acids. These may be substitutions, insertions or deletions but are preferably substitutions. Where deletions are made these are preferably deletion of up to 1, 2, 3, 4, 5, 7 or 10 amino acids, (e.g. deletions from the N or C terminus). Mutants thus include proteins containing amino acid substitutions, e.g. conservative amino acid substitutions that do not affect the function or activity of the protein in an adverse manner. This term is also intended to include natural biological variants (e.g. allelic variants or geographical variations within the species from which the Coversin proteins are derived). Mutants with improved ability to bind wild-type C5 and/or C5 from subjects with a C5 polymorphism (e.g. C5 polymorphisms that render treatment by eculizumab ineffective, or reduce the efficacy of treatment with eculizumab) and/or LTB4 may also be designed through the systematic or directed mutation of specific residues in the protein sequence.

These modifications may be made to the Coversin polypeptide as set out in SEQ ID NO: 2 and SEQ ID NO: 4 and the molecule will remain useful and will be considered to be a functional variant provided that the resulting modified Coversin polypeptide retains LTB4 binding activity and C5 binding comparable with the Coversin polypeptide as set out in SEQ ID NO: 2 and SEQ ID NO: 4, which can be determined e.g. using the tests referred to elsewhere herein (e.g. the binding to each of these is at least 80, 85, 90, 95% of the binding compared to the unmodified Coversin polypeptide).

Given the requirement for functional variants to bind C5 and LTB4, when modification are made, certain residues should be excluded from modification. These include conserved cysteine resides. Other resides should be excluded from modification or, if substituted, should only be subject to conservative modification. These are the LTB4 binding residues and C5 binding residues as defined below. Given that the binding of LTB4 and C5 is relatively well understood it is possible to design a molecule that may have a percentage identity of around 65% to Coversin but in which the changes are confined to residues which are not involved in C5 and LTB4 binding.

In some embodiments each of the six cysteine amino acids at positions 6, 38, 100, 128, 129, 150 of the mature Coversin molecule (e.g. as set out in SEQ ID NO: 4 which corresponds to residues 19 to 168 of the full length protein including the signal sequence) is retained and at least five, ten or fifteen or each of the LTB4 binding residues and at least five, ten or fifteen or twenty or each of C5 binding residues set out below is retained or is subject to a conservative modification.

In some embodiments each of the six cysteine amino acids at positions 6, 38, 100, 128, 129, 150 of SEQ ID NO: 4 is retained and at least five, ten or fifteen or each of the LTB4 binding residues and at least five, ten or fifteen or twenty or each of C5 binding residues set out below is retained or is subject to a conservative modification, wherein up to 2, 3, 4, 5, 10, 15, 20 of the LTB4 and C5 binding residues are subject to a conservative modification. In some embodiments each of the six cysteine amino acids at positions 6, 38, 100, 128, 129, 150 of SEQ ID NO: 4 is retained and at least five, ten or fifteen or each of the LTB4 binding residues and at least five, ten or fifteen or twenty or each of C5 binding residues set out below is retained.

In some embodiments each of the six cysteine amino acids at positions 6, 38, 100, 128, 129, 150 of SEQ ID NO: 4 is retained and each of the LTB4 binding residues and each of C5 binding residues set out below is retained or is subject to a conservative modification.

In some embodiments each of each of the six cysteine amino acids at positions 6, 38, 100, 128, 129, 150 of SEQ ID NO: 4 is retained and each of the LTB4 binding residues and each of C5 binding residues set out below is retained or is subject to a conservative modification, wherein up to 2, 3, 4, 5, 10, 15, 20 of the C5 and/or LTB4 binding residues are subject to a conservative modification.

In some embodiments each of the six cysteine amino acids at positions 6, 38, 100, 128, 129, 150 of SEQ ID NO: 4 is retained and each of the LTB4 binding residues and each of C5 binding residues set out below is retained.

Modifications made outside of these regions may be conservative or non-conservative.

In each of these embodiments the spacing between these six cysteine amino acid residues is preferably retained to preserve the overall structure of the molecule (e.g. the molecule comprises six cysteine residues that are spaced relative to each other at a distance of 32 amino acids apart, 62 amino acids apart, 28 amino acids apart, 1 amino acid apart and 21 amino acids apart as arranged from the amino terminus to the carboxyl terminus of the sequence according to amino acids 1 to 168 of the amino acid sequence in FIG. 2).

### LTB4 Binding Residues

Resides that are thought to be involved in binding to LTB4 and are preferably retained in unmodified form or are subject to conservative changes only in the sequence of any molecule that is modified relative to SEQ ID NO:2 or SEQ ID NO:4 are Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115 (numbering according to SEQ ID NO:4).

### C5 Binding Residues

Resides that are thought to be involved in binding to LTB4 are preferably retained in unmodified form in the sequence of any molecule that is modified relative to SEQ ID NO:2 or SEQ ID NO:4 are Val26, Val28, Arg29, Ala44, Gly45, Gly61, Thr62, Ser97, His99, His101, Met 114, Met 116, Leu117, Asp118, Ala119, Gly120, Gly121, Leu122, Glu123, Val124, Glu125, Glu127, His146, Leu147 and Asp 149 (numbering according to SEQ ID NO:4).

LTB4 and/or C5 Binding Residues

There are two histidine residues involved in both LTB4 and C5 binding, His99 and His101. The list of residues involved in LTB4 and/or C5 binding is therefore Phe18, Tyr25, Val26, Val28, Arg29, Arg36, Leu39, Gly41, Pro43, Ala44, Gly45, Leu52, Val54, Met56, Phe58, Gly61, Thr62, Thr67, Trp69, Phe71, Gln87, Arg89, Ser97, His99, His101, Asp103, Met 114, Trp115, Met 116, Leu117, Asp118, Ala119, Gly120, Gly121, Leu122, Glu123, Val124, Glu125, Glu127, His146, Leu147 and Asp 149 (numbering according to SEQ ID NO:4).

Functional equivalents of Coversin include fragments of the Coversin protein providing that such fragments retain the ability to bind wild-type C5 and/or C5 from subjects with a C5 polymorphism (e.g. C5 polymorphisms that render treatment by eculizumab ineffective, or reduce the efficacy of treatment with eculizumab) and/or LTB4. Fragments may include, for example, polypeptides derived from the Coversin protein sequence (or homologue) which are less than 150 amino acids, less than 145 amino acids, provided that these fragments retain the ability to bind to complement wild-type C5 and/or C5 from subjects with a C5 polymorphism (e.g. C5 polymorphisms that render treatment by eculizumab ineffective, or reduce the efficacy of treatment with eculizumab) and/or LTB4. Fragments may include, for example, polypeptides derived from the Coversin protein sequence (or homologue) which are at least 150 amino acids, at least 145, amino acids, provided that these fragments retain the ability to bind to complement wild-type C5 and/or C5 from subjects with a C5 polymorphism (e.g. C5 polymorphisms that render treatment by eculizumab ineffective or reduce the efficacy of treatment with eculizumab) and/or LTB4.

Any functional equivalent or fragment thereof preferably retains the pattern of cysteine residues that is found in Coversin. For example, said functional equivalent comprises six cysteine residues that are spaced relative to each other at a distance of 32 amino acids apart, 62 amino acids apart, 28 amino acids apart, 1 amino acid apart and 21 amino acids apart as arranged from the amino terminus to the carboxyl terminus of the sequence according to amino acids 1 to 168 of the amino acid sequence in FIG. 2 (SEQ ID NO:2). Exemplary fragments of Coversin protein are disclosed in SEQ ID NO: 4, SEQ ID NO: 6, SEQ ID NO: 8, SEQ ID NO: 10, SEQ ID NO: 12, SEQ ID NO: 14. The DNA encoding the corresponding fragments are disclosed in SEQ ID NO: 3, SEQ ID NO: 5, SEQ ID NO: 7, SEQ ID NO: 9, SEQ ID NO: 11, SEQ ID NO: 13.

Included as such fragments are not only fragments of the O. moubata Coversin protein that is explicitly identified herein in FIG. 2, but also fragments of homologues of this protein, as described above. Such fragments of homologues will typically possess greater than 60% identity with fragments of the Coversin protein sequence in FIG. 2, although more preferred fragments of homologues will display degrees of identity of greater than 70%, 80%, 90%, 95%, 98% or 99%, respectively with fragments of the Coversin protein sequence in FIG. 2. Preferably such fragment will retain the cysteine spacing referred to above. Fragments with improved properties may, of course, be rationally designed by the systematic mutation or fragmentation of the wild type sequence followed by appropriate activity assays. Fragments may exhibit similar or greater affinity for C5, either the wild-type or polymorphic variant of C5 or both, and/or LTB4 as Coversin. These fragments may be of a size described above for fragments of the Coversin protein.

As discussed above, Coversin-type proteins preferably bind to both wild-type C5 and/or C5 from subjects with a C5 polymorphism (e.g. C5 polymorphisms that render treatment by eculizumab ineffective, or reduce the efficacy of treatment with eculizumab) and LTB4.

Any substitutions are preferably conservative substitutions, for example according to the following Table. Amino acids in the same block in the second column and preferably in the same line in the third column may be substituted for each other:

| | | |
|---|---|---|
| Aliphatic | Non-polar | G A P |
| | | I L V |
| | Polar-uncharged | C S T M |
| | | N Q |
| | Polar-charged | D E |
| | | K R |
| Aromatic | | H F W Y |

A functional equivalent used according to the invention may be a fusion protein, obtained, for example, by cloning a polynucleotide encoding the Coversin protein or a functionally equivalent in frame to the coding sequences for a heterologous protein sequence. The term “heterologous”, when used herein, is intended to designate any polypeptide other than the Coversin protein or its functional equivalent. Examples of heterologous sequences that can be comprised in the soluble fusion proteins either at N- or at C-terminus, are the following: extracellular domains of membrane-bound protein, immunoglobulin constant regions (Fc region), PAS or XTEN or similar unstructured polypeptides, multimerization domains, domains of extracellular proteins, signal sequences, export sequences, or sequences allowing purification by affinity chromatography. Many of these heterologous sequences are commercially available in expression plasmids since these sequences are commonly included in the fusion proteins in order to provide additional properties without significantly impairing the specific biological activity of the protein fused to them [35]. Examples of such additional properties are a longer lasting half-life in body fluids (e.g. resulting from the addition of an Fc region or PASylation [36]), the extracellular localization, or an easier purification procedure as allowed by a tag such as a histidine, GST, FLAG, avidin or HA tag. Fusion proteins may additionally contain linker sequences (e.g. 1-50 amino acids in length, such that the components are separated by this linker.

Fusion proteins are thus examples of proteins comprising a Coversin-like protein, and include by way of specific example a protein comprising a PAS sequence and a Coversin-type protein sequence. PAS sequences are described e.g. in [36], and EP2173890, with a PASylated Coversin molecule being described in Kuhn et al [37]. PASylation describes the genetic fusion of a protein with conformationally disordered polypeptide sequences composed of the amino acids Pro, Ala, and/or Ser. This is a technology developed by XL Protein (http://xl-protein.com/) and provides a simple way to attach a solvated random chain with large hydrodynamic volume to the protein to which it is fused. The polypeptide sequence adopts a random coil structure. The apparent molecular weight of the resulting fusion protein is thus much larger than the actual molecular weight of the fusion protein. This greatly reduces clearance rates by kidney filtration in biological systems. Appropriate PAS sequences are described in EP2173890, as well as [36]. Any suitable PAS sequence may be used in the fusion protein. Examples include an amino acid sequence consisting of at least about 100 amino acid residues forming a random coil conformation and consisting of or consisting essentially of alanine, serine and proline residues (or consisting of or consisting essentially of proline and alanine residues). This may comprise a plurality of amino acid repeats, wherein said repeats consist of or consist essentially of Ala, Ser, and Pro residues (or proline and alanine residues) and wherein no more than 6 consecutive amino acid residues are identical. Proline residues may constitute more than 4% and less than 40% of the amino acids of the sequence. The sequence may comprise an amino acid sequence selected from:

(SEQ ID NO: 15)
ASPAAPAPASPAAPAPSAPA;
(SEQ ID NO: 16)
AAPASPAPAAPSAPAPAAPS;
(SEQ ID NO: 17)
APSSPSPSAPSSPSPASPSS,
(SEQ ID NO: 18)
SAPSSPSPSAPSSPSPASPS,
(SEQ ID NO: 19)
SSPSAPSPSSPASPSPSSPA,
(SEQ ID NO: 20)
AASPAAPSAPPAAASPAAPSAPPA
and
(SEQ ID NO: 21)
ASAAAPAAASAAASAPSAAA

or circular permuted versions or multimers of these sequences as a whole or parts of these sequences. There may, for example be 5-40, 10-30, 15-25, 18-20 preferably 20-30 or 30 copies of one of the repeats present in the PAS sequence, i.e. one of SEQ ID NOs 15-21, preferably 15. Preferably the PAS sequence comprises or consists of 30 copies of SEQ ID NO:15. Preferably the PAS sequence is fused to the N terminus of the Coversin-type protein (directly or via a linker sequence) and in certain preferred embodiments the Coversin-type protein may comprise or consist of amino acids 19-168 of SEQ ID NO:2 (e.g. the fusion protein comprises (a) a PAS sequence consisting of 30 copies of SEQ ID NO:15 and (b) amino acids 19-168 of SEQ ID NO:2, wherein (a) is fused to the N terminus of (b) directly or via a linker sequence). An exemplary sequence is provided in FIG. 5 and SEQ ID NO:22.

Fusion proteins may additionally contain linker sequences (e.g. 1-50, 2-30, 3-20, 5-10, 2-4, 3-5, 1, 2, 3, 4, 5, 6, 7, 8, 9, 10 or up to 1, 2, 3, 4, 5, 6, 7, 8, 9, 10 amino acids in length), such that the components are separated by this linker. In one embodiment the linker sequence can be a single alanine residue.

In the present “PAS-Coversin” is intended to refer to a functional equivalent of Coversin that is PASylated, e.g. as described above. The precise sequence of the tested PAS-Coversin molecule in Examples 1 and 2 is set out in FIG. 5 and SEQ ID NO:22. PAS-Coversin has the advantage that its longer half-life allows less frequent administration, which is more convenient for patients. PAS-Coversin thus combines the advantages of Coversin, in that it inhibits both the C5 and the LTB4 dependent pathways, yet can be administered less frequently than Coversin thus providing an administration advantage.

The protein and functional equivalents thereof, may be prepared in recombinant form by expression in a host cell. Such expression methods are well known to those of skill in the art and are described in detail by [38] and [39]. Recombinant forms of the Coversin protein and functional equivalents thereof are preferably unglycosylated. Preferably the host cell is E. coli.

The Coversin protein and functional equivalents thereof, are preferably in isolated form, e.g. separated from at least one component of the host cell and/or cell growth media in which it was expressed. In some embodiments, the Coversin protein or functional equivalent thereof is purified to at least 90%, 95%, or 99% purity as determined, for example, by electrophoresis or chromatography. The proteins and fragments of the present invention can also be prepared using conventional techniques of protein chemistry. For example, protein fragments may be prepared by chemical synthesis. Methods for the generation of fusion proteins are standard in the art and will be known to the skilled reader. For example, most general molecular biology, microbiology recombinant DNA technology and immunological techniques can be found in [38] or [40].

According to a further embodiment of the invention, the agent may be a nucleic acid molecule encoding the Coversin-type protein. For example, gene therapy may be employed to effect the endogenous production of the Coversin-type protein by the relevant cells in the subject, either in vivo or ex vivo. Another approach is the administration of “naked DNA” in which the therapeutic gene is directly injected into the bloodstream or into muscle tissue.

Preferably, such a nucleic acid molecule comprises or consists of bases 55 to 507 of the nucleotide sequence in FIG. 2 (SEQ ID NO: 1). This nucleotide sequence encodes the Coversin protein in FIG. 2 without the signal sequence. The first 54 bases of the nucleotide sequence in FIG. 2 encode the signal sequence which is not required for complement inhibitory activity or LTB4 binding activity. Alternatively, the nucleic acid molecule may comprise or consist of bases 1 to 507 of the nucleic acid sequence in FIG. 2, which encodes the protein with the signal sequence.

### Modes of Administration

Coversin-type proteins do not require a medical professional for administration to be carried out, and these molecules are rapidly absorbed. In contrast, many recombinant antibodies are absorbed very slowly or cannot be administered by subcutaneous injection or other routes of administration and as a result need to be infused over long periods (e.g. intravenously). The administration of such molecules therefore requires a medical professional. Thus, Coversin-type proteins also possess the advantage of being easier to administer than other agents that require infusion.

The agent is administered in a therapeutically or prophylactically effective amount. The term “therapeutically effective amount” refers to the amount of agent needed to treat the rheumatic disease. In this context, “treating” includes reducing the severity of the disorder.

The term “prophylactically effective amount” used herein refers to the amount of agent needed to prevent the relevant condition, e.g. rheumatic disease. In this context, “preventing” includes reducing the severity of the disorder, e.g. if the presence of the disorder is not detected before the administration of the agent is commenced. Reducing the severity of the disorder could be, for example, reducing the levels of pain, inflammation, joint stiffness or fever.

The reduction or improvement is relative to the outcome without administration or the agent as described herein. The outcomes are assessed according to the standard criteria used to assess such patients. To the extent that this can be quantitated, there is a reduction or improvement of at least 10, 20, 30, 40, 50, 60, 70, 80, 90, 100% in the relative criteria (e.g. levels of pain, inflammation, joint stiffness or fever).

Preferably, the dose, calculated on the basis of the Coversin molecule is from 0.1 mg/kg/day to 10 mg/kg/day (mass of drug compared to mass of patient), e.g. 0.2-5, 0.25-2, or 0.1-1 mg/kg/day. As fusion proteins (e.g. as discussed herein) are larger than the Coversin molecule an equivalent molar amount could be used for such proteins. Thus for a functional equivalent of Coversin, an equivalent molar amount of the dose referred to above can be used. For example for a fusion protein comprising Coversin and a PAS portion of about 600 amino acids, or a PAS portion as defined herein, e.g. PAS-Coversin) an equivalent molar amount of 0.1 mg/kg/day is 0.4 mg/kg/day, so the dose could be 0.4 mg/kg/day to 40 mg/kg/day (mass of drug compared to mass of patient), e.g. 0.8-20, 1-8, or 0.4-4 mg/kg/day. Alternatively, and to account for the longer half-life of these fusion proteins, greater amounts can be given per dose, and the dose administered less often, e.g. 40 mg-2 g, 50 mg-1.5 g, 75 mg-1 g, over the course of one week, e.g. with administration being e.g. one or twice per week.

The therapeutically or prophylactically effective amount can additionally be defined in terms of the inhibition of terminal complement, for example, an amount that means that terminal complement activity (TCA) is reduced by at least 10, 20, 30, 40, 50, 60, 70, 80, 90, 91, 92, 93, 94, 95, 96, 97, 98, 99, 100%, compared to terminal complement activity in the absence of treatment. Dose and frequency may be adjusted in order to maintain terminal complement activity at the desired level, which may be, for example 10% or less, for example 9, 8, 7, 6, 5, 4, 3, 2, 1% or less compared to terminal complement activity in the absence of treatment.

The therapeutically or prophylactically effective amount can additionally be defined in terms of the reduction of LTB4 levels in plasma, for example, an amount that means that the LTB4 level in plasma is reduced by at least 10, 20, 30, 40, 50, 60, 70, 80, 90, 91, 92, 93, 94, 95, 96, 97, 98, 99, 100%, compared to the LTB4 level in plasma in the absence of treatment or which causes LTB4 levels to be within a certain range of the normal levels (e.g. 90-110% of normal, 85-115% of normal). Dose and frequency may be adjusted in order to maintain the LTB4 level in plasma at the desired level, which may be, for example 90%, 80%, 70%, 60%, 50%, 40%, 30%, 20%, 10% or less, for example 9, 8, 7, 6, 5, 4, 3, 2, 1% or less compared to the LTB4 level in plasma in the absence of treatment or which is within a certain range of the normal levels (e.g. 90-110% of normal, 85-115% of normal). LTB4 levels may be determined by routine methods (e.g. immunoassays, see e.g. the commercially available R&D Systems assay based on a sequential competitive binding technique [41]).

Where a dose is given, this relates to a dose of the agent which is a protein or functional equivalent thereof. Appropriate doses for an agent which is a nucleic acid molecule may be used to give rise to these levels. Doses may vary to account for the presence of non-active protein present (e.g. PAS-Coversin with a 600 amino acid PAS portion has approximately 4× the molecular weight of Coversin so an equivalent molar amount would be approximately four times the amount of Coversin). An equivalent molar amount of any dose provided for Coversin may be used for any Coversin functional equivalent thereof which contains additional sequence. The equivalent molar amount can be calculated using routine methods.

Terminal complement activity can be measured by standard assays known in the art, e.g. using the Quidel CH₅₀ haemolysis assay and the sheep red blood cell lytic CH50 assay.

The frequency with which the dose needs to be administered will depend on the half-life of the agent involved. The Coversin protein or a functional equivalent thereof, may be administered e.g. on a twice daily basis, daily basis, or every two, three, four, five, six, or seven, days or more e.g. twice daily or on a daily basis). Extended half-life versions, e.g. PASylated Coversin molecules could be administered less frequently (e.g. every two, three, four days, five, six, seven, 10, 15 or 20 days or more, e.g. once daily or every two or more days, or every week).

The exact dosage and the frequency of doses may also be dependent on the patient's status at the time of administration. Factors that may be taken into consideration when determining dosage include the need for treatment or prophylaxis, the severity of the disease state in the patient, the general health of the patient, the age, weight, gender, diet, time and frequency of administration, drug combinations, reaction sensitivities and the patient's tolerance or response to therapy. The precise amount can be determined by routine experimentation, but may ultimately lie with the judgement of the clinician.

The dosage regimen may also take the form of an initial “ablating regimen” followed by one or more subsequent doses (e.g. maintenance dose). In general, the ablating regimen will be greater than the subsequent dose(s). By way of example for Coversin this may be an ablating regimen of 0.6-1.2 mg/kg, then 0.3-0.6 mg/kg for 8-18, 10-14, or 11-13 hours (e.g. about 12 hours) later, followed by a maintenance dose of 0.45-0.9 mg/kg, which may be administered e.g. once daily.

For PASylated versions (e.g. PAS-Coversin, e.g. as described elsewhere herein) a suitable regimen may be an ablating regimen of 6-12 mg/kg (e.g. 600 mg), then 6-12 mg/kg (e.g. 600 mg) 3-10, 4-8, 5-7, e.g. about 7 days later, followed by a maintenance dose of 4-8 mg/kg (e.g. 400 mg), which may be administered e.g. once daily.

The ablating dose or doses may be at least 1.5, 2, or 5 times greater than the maintenance dose. The ablating dose may be administered as a single dose, or as one or more doses in a particular time frame (e.g. two doses). Typically, the loading dose will be 1, 2, 3, 4 or 5 doses administered in a single 24 hour period (or a single week for an extended half-life version). The maintenance dose may be a lower dose that is repeated at regular intervals. The maintenance dose may be repeated at intervals, such as every 12, 24, or 48 hours (or every week, or every two weeks for an extended half-life version). The precise regimen can be determined by routine experimentation, but may ultimately lie with the judgement of the clinician. The maintenance dose may be at least 20, 30, 40, 50, 60, 70, 80, 90 or 100% of the initial ablating dose, or up to 20, 30, 40, 50, 60, 70, 80, 90 or 100% of the initial ablating dose.

In a further embodiment the same dose is used throughout the course of treatment (e.g. daily or twice daily or weekly).

The agent will generally be administered in conjunction with or in a pharmaceutically acceptable carrier. The term “pharmaceutically acceptable carrier”, in general will be a liquid or but may include other agents provided that the carrier does not itself induce toxicity effects or cause the production of antibodies that are harmful to the individual receiving the pharmaceutical composition. Pharmaceutically acceptable carriers may e.g. contain liquids such as water, saline, glycerol, ethanol or auxiliary substances such as wetting or emulsifying agents, pH buffering substances and the like. The pharmaceutical carrier employed will thus vary depending on the route of administration. A thorough discussion of pharmaceutically acceptable carriers is available in [42]. In a preferred embodiment the agent is administered in a liquid, e.g. in a solution in water or PBS.

The agent may optionally be delivered using colloidal delivery systems (e.g. liposomes, nanoparticles or microparticles (e.g. as discussed in [43])). Advantages of these carrier systems include protection of sensitive proteins, prolonged release, reduction of administration frequency, patient compliance and controlled plasma levels.

Liposomes (e.g. comprising phospholipids of synthetic and/or natural origin) may e.g. be 20 nm 100 or 200 micrometers, e.g. small unilamellar vesicles (25-50 nm), large unilamellar vesicles (100-200 nm), giant unilamellar vesicles (1-2 μm) or multilamellar vesicles (MLV; 1 μm-2 μm).

Nanoparticles (colloidal carriers with size ranging from 10 to 1000 nm) can be fabricated from lipids, polymers or metal. Polymeric nanoparticles may be made from natural or synthetic polymers (e.g. chitosan, alginate, PCL, polylactic acid (PLA), poly (glycolide), PLGA) and may be generated as nanospheres (molecules are uniformly distributed into polymeric matrix) or nanocapsules (carrying drug molecules confined within a polymeric membrane).

Microparticles e.g. made of starch, alginate, collagen, poly (lactide-co-glycolide) (PLGA), polycaprolactones (PCL) can also be used.

Hydrogels may alternatively or additionally be present.

For larger molecular weight molecules, e.g. fusion proteins additional excipients such as hyaluronidase may also be used, e.g. to allow administration of larger volumes (e.g. 2-20 ml).

The agent is preferably delivered by subcutaneous injection or injection into the synovial joint fluid. Subcutaneous injection is preferred in view of the ease of administration for the subject. In some embodiments this is via once or twice daily subcutaneous injection.

Preferably the course of treatment is continued for at least 1, 2, 3, 4, 5 or 6 weeks, or at least 1, 2, 3, 4, 5 or 6 months or at least 1, 2, 3, 4, 5 or 6 years. The course of treatment is preferably continued at least until the subject's symptoms have reduced. The course of treatment may thus be administration of the agent (e.g. daily, every other day or weekly) for at least 1, 2, 3, 4, 5, 6, 7, 8, 9, 10, 15, 20, 25, 30, 35, 40 weeks.

The maintenance dose (e.g. a single daily or weekly maintenance dose) may remain constant throughout the course of treatment) or the maintenance dose (e.g. a daily maintenance dose) may be modified (e.g. increased or decreased) during the course of treatment. The maintenance dose may be modified in order to maintain terminal complement activity and plasma LTB4 levels at a desired level, e.g. terminal complement activity at 10% or less compared to serum from said patient in the absence of treatment or compared to normal control serum and/or plasma LTB4 levels at 90% or less compared to plasma from said patient in the absence of treatment, or to attain plasma LTB4 levels that are within a certain range of the normal levels (e.g. 90-110% of normal, 85-115% of normal). The or each maintenance dose may be continued for at least 1, 2, 3, 4, 5, 6, 7, 8, 9, 10 weeks, e.g. daily for at least 1, 2, 3, 4, 5, 6, 7, 8, 9, 10 weeks. The maintenance dose may be decreased as the subject's symptoms improve. The amount of agent or the frequency with which the agent is administered may be decreased as the subject's symptoms improve.

There may thus be an initial ablating dose or regimen, followed by an initial maintenance dose (e.g. a daily or weekly initial maintenance dose) which may be a maintenance dose as defined above, and one or more further maintenance doses (e.g. a daily or weekly further maintenance dose), e.g. at least 2, 3, 4, 5 further maintenance doses.

The invention thus further comprises a method of treating or preventing a rheumatic disease in a subject, comprising administering to the subject an initial ablating dose or regimen of the agent as defined above, and then administering maintenance doses (e.g. daily or weekly maintenance doses) of the agent as defined above, wherein there is an initial maintenance dose and one or more further maintenance doses.

The invention thus further comprises an agent as defined above for use in a method of treating or preventing a rheumatic disease in a subject, the method comprising administering to the subject an initial ablating dose or regimen of the agent as defined above, and then administering maintenance doses (e.g. daily or weekly maintenance doses) of the agent as defined above, wherein there is an initial maintenance dose and one or more further maintenance doses.

The one or more further maintenance doses may be determined by testing the terminal complement activity in the subject (e.g. in a biological sample from the subject) or plasma LTB4 level, and determining the further maintenance dose on the basis of the level of terminal complement activity and/or plasma LTB4 level and/or testing the subject's symptoms and determining the further maintenance dose on the basis of the symptoms. The method may optionally further comprise administering said further maintenance dose. Said further dose may be calculated to be at a level that maintains terminal complement activity at the desired level.

Where a biological sample is taken, this may be blood, e.g. a whole blood, plasma or a serum sample. The method optionally further comprises the step of taking the sample, and further optionally comprises the step of determining the TCA of the sample and/or the step of determining the plasma LTB4 level.

The one or more further maintenance doses may be determined by testing the terminal complement activity in the subject (e.g. in a biological sample) and/or plasma LTB4 level, and determining the further maintenance dose on the basis of the level of terminal complement activity and/or plasma LTB4 level, and/or testing the subject's symptoms and determining the further maintenance dose on the basis of the symptoms. The method may optionally further comprise administering said further maintenance dose. Said further dose may be calculated to be at a level that maintains terminal complement activity and/or plasma LTB4 level at the desired level.

In certain aspects, the desired complement activity level is 10% or less compared to serum from said subject in the absence of treatment or compared to normal control serum and/or plasma LTB4 level is 90% or less compared to plasma from said patient in the absence of treatment, and/or plasma LTB4 levels are within a certain range of the normal levels (e.g. 90-110% of normal, 85-115% of normal).

In certain aspects, if the TCA and/or plasma LTB4 is higher than the desired level the maintenance dose is increased, and optionally wherein if TCA is less than 5, 4, 3, 2, 1% and/or LTB4 plasma levels are 90% or less compared to plasma from said patient in the absence of treatment (or plasma LTB4 levels are within a certain range of the normal levels (e.g. 90-110% of normal, 85-115% of normal) the dose is maintained or decreased.

In certain aspects, if the symptoms deteriorate the maintenance dose is increased, and optionally wherein if the symptoms improve the dose is maintained or decreased.

In some embodiments the subject is tested within one month of initiating the treatment, within two weeks of initiating the treatment, within a week of initiating the treatment. In other embodiments the subject is tested once a day or at least once a day, once a week, or at least once a week, once every two weeks or at least once every two weeks, once a month or once every two months.

The dosage regimen may also take the form of fixed dose not dependent on the weight of the subject being treated. The fixed dose may be administered as a single dose, or as one or more doses in a particular time frame. The fixed dose can be 1 mg-100 mg of Coversin (e.g. SEQ ID NO: 4) for typical human patients (e.g. those between 50 kg and 100 kg in weight). The molecular weight of Coversin-type proteins can be used to calculate equivalent fixed doses of functionally equivalent agents. In some embodiments, the fixed dose is between 1 mg-80 mg, 1 mg-50 mg, 5 mg-80 mg, 5 mg-50 mg, 10 mg-60 mg, 10 mg-50 mg, 20 mg-50 mg, 20 mg-40 mg or 25 mg-35 mg of Coversin (e.g. SEQ ID NO: 4) or the molar equivalent of a Coversin-type protein. Preferably the fixed dose is 30 mg, or 45 mg of Coversin (SEQ ID NO: 4) or the molar equivalent of a Coversin-type protein. Typically, the fixed dose will be 1, 2, 3, 4 or 5 doses administered in a single 24 hour period. The fixed dose may be repeated at intervals, such as every 3, 4, 6, 8, 12, 24, or 48 hours. The precise regimen can be determined by routine experimentation, but may ultimately lie with the judgement of the clinician.

### BRIEF DESCRIPTION OF FIGURES

FIG. 1: Schematic diagram of classical and alternative pathways of complement activation. Anaphylatoxins are enclosed in starbursts.

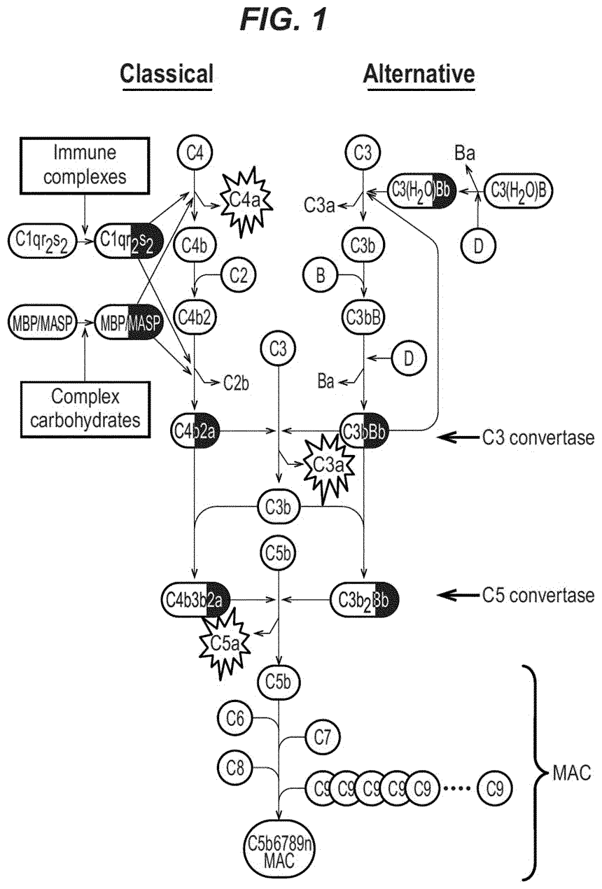

FIG. 2A: Primary sequence of Coversin. Signal sequence underlined. Cysteine residues in bold type. Nucleotide and amino acid number indicated at right. The nucleotide sequence is SEQ ID NO: 1 and the amino acid sequence is SEQ ID NO: 2.

FIG. 2B: Examples of Coversin variants.

FIG. 3: Clinical score and paw width of experimental RA in vehicle versus mice treated with prophylactic Coversin, PAS-Coversin or Zileuton.

FIG. 4: Clinical score of experimental RA in vehicle versus mice treated with PAS-Coversin demonstrates ameliorated disease in mice treated with PAS-Coversin compared to the vehicle control group, Zileuton, and PAS-L Coversin, which does not bind C5.

FIG. 5: PAS-Coversin sequence.

### EXAMPLES

### Example 1—Effect of Coversin in K/B×N Serum Transfer Model (Prophylactic)

The K/B×N serum-transfer arthritis model was used to test whether development of arthritis could be prevented by Coversin. This model is a well-known murine model in which the immunological mechanisms occurring in rheumatoid arthritis (RA) are induced by transferring serum from arthritic transgenic K/B×N mice to naive mice. In the absence of treatment arthritic symptoms occur a few days later. This model is recognised to be highly relevant for RA, especially for the preclinical screening of new therapeutic targets for RA and perhaps other forms of inflammatory arthritis [44].

Experimental RA was induced using the protocol described by [45]. Five mice were tested in each treatment group.

On day 0 C57BL/6 mice were assessed for clinical score and paw thickness using standard protocols [45]. All mice received an injection of K/B×N serum on day 0 and on day 2. Each treatment was administered every day from day 0 to day 14. On days where serum was also injected (day 0 and day 2) drug was administered immediately after the serum injection. Clinical score and paw thickness were measured every other day.

Treatment groups were as follows:

Group 1: PBS (vehicle, subcutaneous, q24h)

Group 2: Treatment (Coversin 5 mg/kg subcutaneous, q12h)

Group 3: Treatment (Zileuton 50 mg/kg orally once daily)

Group 4: Treatment (PAS-Coversin 20 mg/kg subcutaneous, once daily)

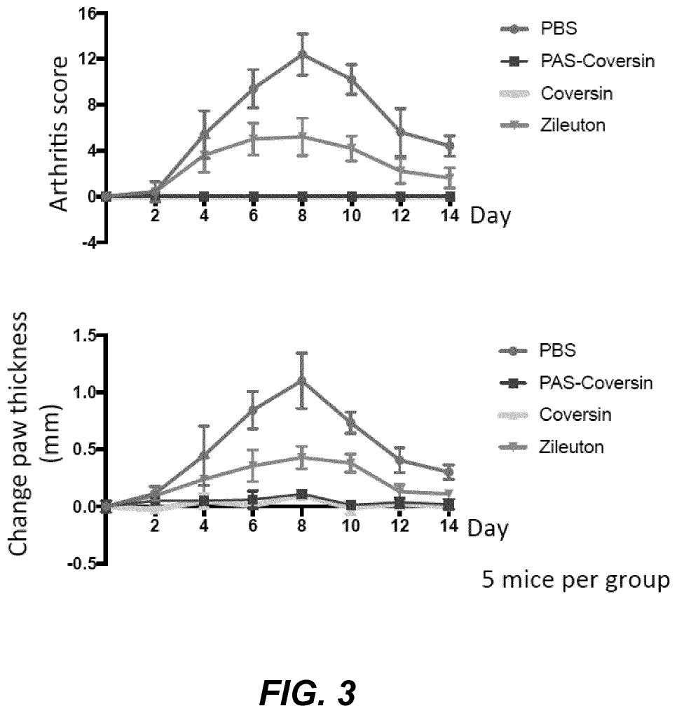

The results of Experiment 1 are shown in FIG. 3. It can be seen that Coversin (q12h) & PAS-Coversin (q24h) completely inhibit the development of arthritis in this model. Zileuton (5-LOX inhibitor) partially ameliorates the development of arthritis in this model.

Coversin and PAS-Coversin reduced the development of arthritis in this model to a greater degree than N-[1-(1-benzothien-2-yl)ethyl]-N-hydroxyurea (Zileuton), which is a 5-lipoxygenase inhibition (5-LOX) oral inhibitor (an important enzyme of the arachidonic acid cascade and is involved in the formation of bioactive leukotrienes (LTs)).

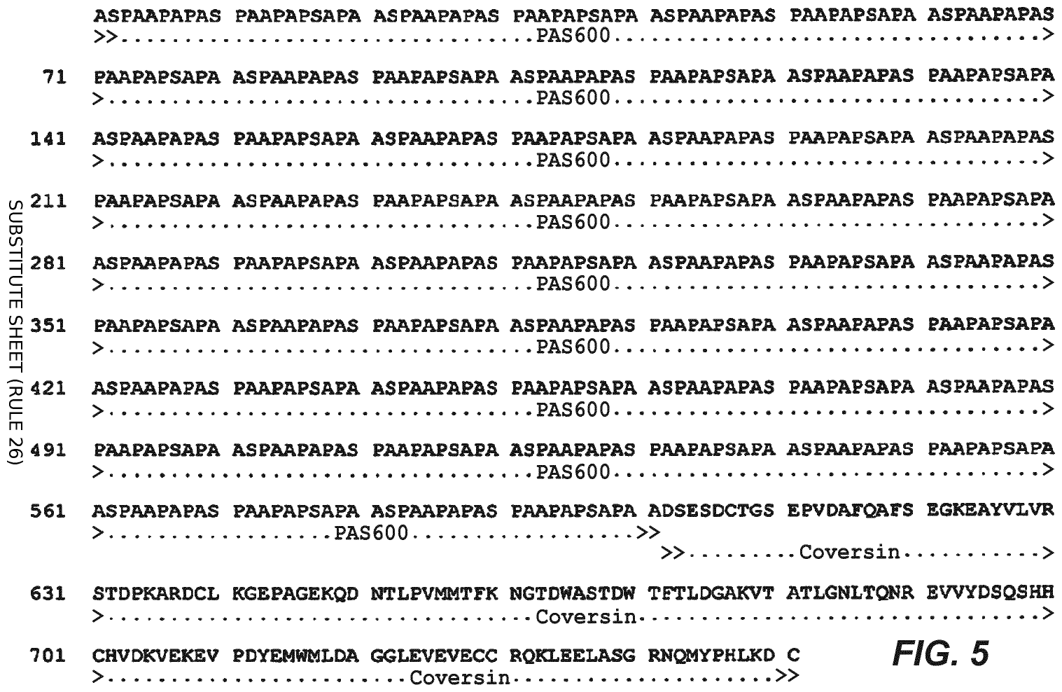

The difference between PAS-Coversin and Coversin is that the PAS-Coversin is “PASylated”. This describes the genetic fusion of a protein with conformationally disordered polypeptide sequences composed of the amino acids Pro, Ala, and/or Ser. This is a technology developed by XL Protein (http://xl-protein.com/) and provides a simple way to attach a solvated random chain with large hydrodynamic volume to the protein to which it is fused. The polypeptide sequence adopts a random coil structure resulting in a large increase in apparent molecular weight and a reduced rate of clearance by kidney filtration. The sequence of PAS-Coversin used in this experiment is shown in FIG. 5 and SEQ ID NO:22.

Because of the higher molecule weight of the PAS-Cov, 20 mg/kg PAS-Cov corresponds to 5 mg/kg Coversin, so in this experiment equivalent doses of the active Coversin agents are administered each day, but Coversin is administered twice as frequently (12 hourly). PAS-Coversin was as effective as Coversin in this experiment but with less frequent administration. Thus, there may be advantages in using longer half-life versions of Coversin, such as PAS Coversin therapeutically.

### Example 2 Effect of Coversin in K/B×N Serum Transfer Model (Treatment)

The K/B×N serum-transfer arthritis (STA) model was used to test whether established arthritic disease could be ameliorated by Coversin.

Experimental RA was induced using the protocol described by [45]. Five mice were tested in each treatment group.

On day 0 C57BL/6 mice were assessed for clinical score and paw thickness using standard protocols [45]. All mice received an injection of K/B×N serum on day 0 and on day 2. The treatment was administered every day from day 4 to day 14. On days where serum was also injected (day 0 and day 2) drug was administered immediately after the serum injection. Clinical score and paw thickness were measured every other day.

Treatment groups were as follows:

Group 1: PBS (vehicle)

Group 2: Treatment (PAS-L Coversin 20 mg/kg subcutaneous once daily)

Group 3: Treatment (Zileuton 50 mg/kg orally once daily)

Group 4: Treatment (PAS-Coversin 20 mg/kg subcutaneous once daily)

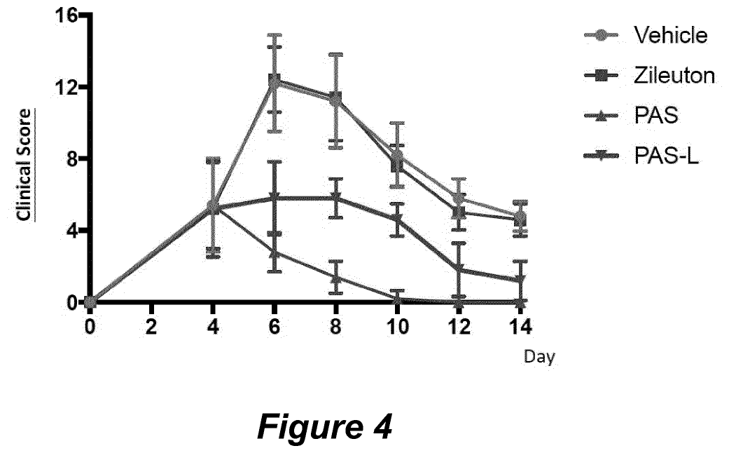

The results of the clinical scores from the second experiment are shown in FIG. 4. It can be seen mice developed arthritis following serum injection. In the vehicle and Zileuton groups maximum clinical score was seen at day 6 of the experiment. Following administration of PAS-L-Coversin (q24h) and PAS-Coversin (q24h), the clinical score decreased for the PAS-Coversin and PAS-L-Coversin groups. For the PAS-L-Coversin group the clinical score was significantly lower than the vehicle and Zileuton treated groups.

The difference between PAS-Coversin and PAS-L-Coversin is that in the L-Coversin molecule the Coversin sequence has been mutated such that it binds LTB4 but does not bind C5 (referred to as “L-Coversin”). The sequence of the L-Coversin sequence is a variant of the mature Coversin sequence (SEQ ID NO: 4) in which the following residues have been modified: Ala44 to Asn, Met116 to Gln, Leu117 to Ser, Gly121 to Ala, Leu122 to Asp, Glu123 to Ala and Asp149 to Gly, (referred to as variant 2, sequence is dsesdctgse pvdafqafse gkeayvlvrs tdpkardclk gepNgekqdn tlpvmmtfkn gtdwastdwt ftldgakvta tlgnitqnre vvydsqshhc hvdkvekevp dyemwQSdag ADAveveccr qkleelasgr nqmyphlkGc (SEQ ID NO:23), where the changes relative to the native Coversin sequence of SEQ ID NO:4 are in capitals).

The PAS-L-Coversin was not as effective as the PAS-Coversin in ameliorating RA but was more effective than Zileuton. This suggests that the dual inhibitory activity of Coversin (C5 and LTB4 inhibition) provides improved therapeutic benefit in this model. It had been expected that combined inhibition of C5 activation and LTB4 would be only as effective in the therapeutic model as LTB4 inhibition alone. However unexpectedly combined inhibition of C5 and LTB4 by Coversin, as shown by the improved effected of Coversin compared to L-Coversin, proved much more effective in the therapeutic model than LTB4 inhibition alone.

### REFERENCES

[1] Hoover et al, 1984, Proc. Nat. Acad. Sci. U.S.A. 81, 2191-2193
[2] Harrison and Murphy, 1995, J. Biol. Chem. 270, 17273-17276
[3] Ford-Hutchinson, 1990, Crit. Rev. Immunol. 10, 1-12
[4] Showell et al., 1995, J. Pharm. Exp. Ther. 273, 176-184
[5] Klaas et al, 2005 J. Exp. Med. 201, 1281-1292
[6] Del Prete et al, 2007 Blood, 109, 626-631
[7] Miyahara et al, 2006 Allergol Int. 55, 91-7
[8] Taube et al, 2006 J. Immunol. 176, 3157-3164
[9] Yamaoka et al, 1989 J. Immunol. 143, 1996-2000
[10] Yokomizo et al, 1997 Nature 387, 620-624
[11] Yokomizo et al, 2000 J. Exp. Med. 192, 421-432
[12] Tager and Luster, 2003 Prostaglandins Leukot. Essent. Fatty Acids 69, 123-134
[13] Yokomizo et al., 2001, J. Biol. Chem. 276, 12454-12459
[14] Kim, N. D. and Luster, A. D. (2007) The Scientific World Journal 7, 1307-1328.
[15] Kim et al., 2006. J. Exp. Med. 203, 829-835
[16] Noiri et al., 2000 Proc Nat Acad Sci USA 97, 823-828
[17] Lundeen et al., 2006. J. Immunol. 177, 3439-3447
[18] Shao et al, 2006. J. Immunol. 176, 6254-6261
[19] Chen et al., 2006. J. Exp. Med. 203, 837-842
[20] Sebaldt et al., 1990 Proc Natl Acad Sd. U.S.A. 8, 6974-6978
[21] Curry et al., 2005 Journal of the American Animal Hospital Association 41, 298-309
[22] Dube et al., 1998. Zileuton: the first leukotriene inhibitor for use in the management of chronic asthma. In: Drazen J M, Dahlen S, Lee T H, eds. Five-lipoxygenase Products in Asthma. New York, N.Y.: Marcel Dekkar, Inc
[23] Sharma and Mohammed, 2006 Immunopharmacology 14, 10-16
[24] GBD 2015 Disease and Injury Incidence and Prevalence, Collaborators. Lancet. 388 (10053): 1545-1602. (2016)
[25] GBD 2013 Mortality and Causes of Death, Collaborators (17 Dec. 2014). Lancet. 385 (9963): 117-71.
[26] WO2004/106369
[27] Jore, M. M. et al, Nature Structural & Molecular Biology 2016 volume 23, pages 378-386
[28] Pincus et al Rheumatology 2008; 47:345-349
[29] Oliver J et al Arthritis Res Ther. 2009; 11(3): 223.
[30] Roversi, P et al Journal of Biological Chemistry 2013, 288(26) 18789-18802
[31] Guo, R. F. and P. A. Ward, Annu Rev Immunol, 2005, 23: p. 821-52
[32] Ricklin D & Lambris J, Nature Biotechnology, 25: 1265-1275 (2007)
[33] Nishimura, J et al., New Engl J. Med., 30; 7: 632-639 (2014)
[34] Breustedt D. A., Schonfeld D. L., Skerra A. (2006) Comparative ligand-binding analysis of ten human lipocalins. Biochim Biophys Acta 1764(2):161-173.
[35] Terpe K, Appl Microbiol Biotechnol, 60: 523-33, 2003
[36] Schlapschy M, et al Protein Eng Des Sel. 2013 August; 26(8):489-501
[37] Kuhn et al Bioconjugate Chem., 2016, 27 (10), pp 2359-2371
[38] Sambrook et al (2000)
[39] Fernandez & Hoeffler (1998)
[40] Ausubel et al. (1991)
[41] https://resources.rndsystems.com/pdfs/datasheets/kge006b.pdf
[42] Remington's Pharmaceutical Sciences; Mack Pub. Co., N.J. 1991
[43] Patel et al Ther. Deliv. (2014) 5(3), 337-365
[44] Christensen, A. et al. Front Immunol. 2016; 7: 213.
[45] Kim et al JEM 2006; 203(4) 829-835

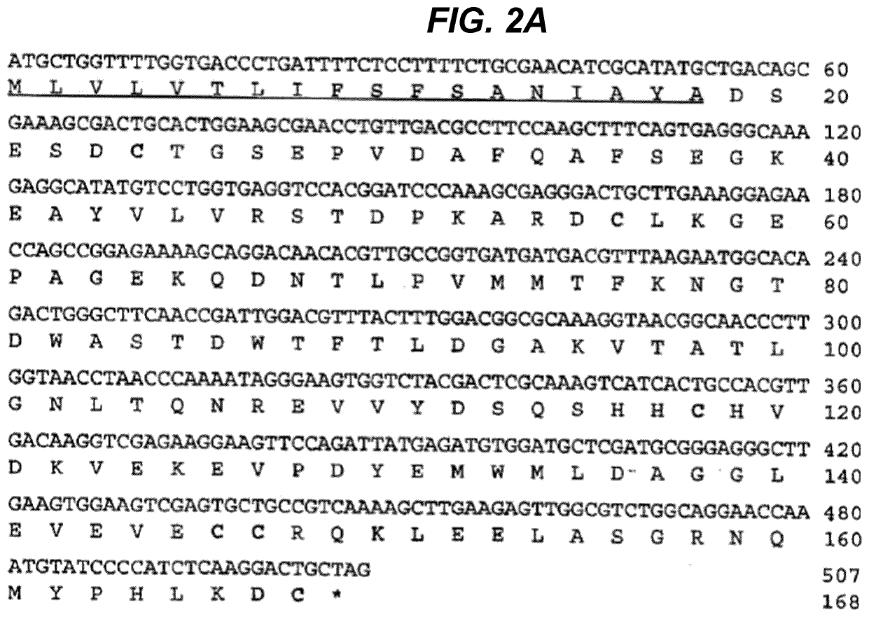

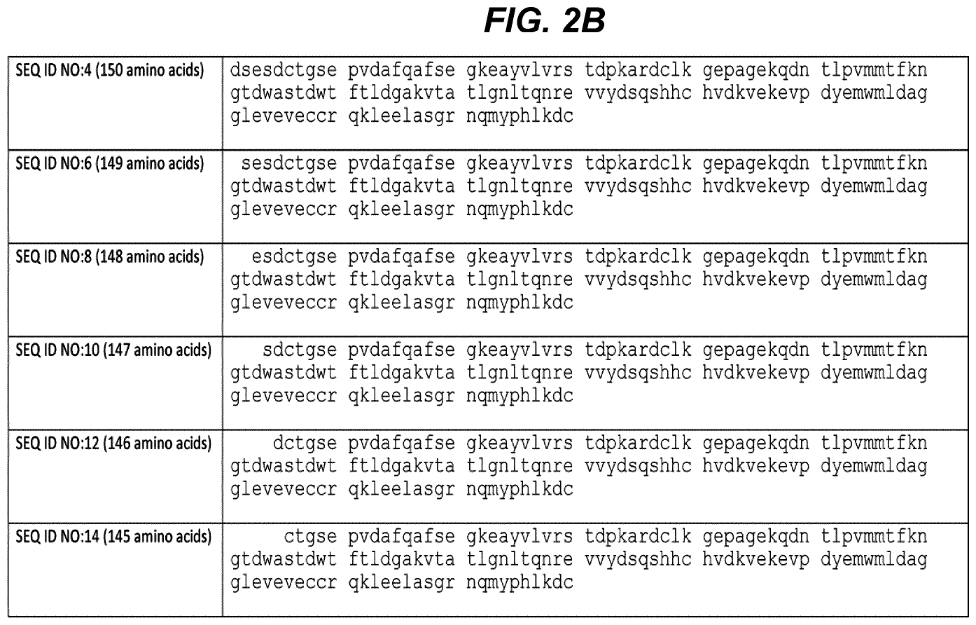

1. A method of treating or preventing a rheumatic disease in a subject, which comprises administering to the subject a therapeutically or prophylactically effective amount of an agent which is a protein comprising amino acids 19 to 168 of the amino acid sequence in FIG. 2 (SEQ ID NO: 2) or a functional equivalent of this protein.

2. An agent which is a protein comprising amino acids 19 to 168 of the amino acid sequence in FIG. 2 (SEQ ID NO: 2) or a functional equivalent of this protein for use in a method of treating or preventing a rheumatic disease in a subject.

3. A method of treating or preventing a rheumatic disease in a subject, which comprises administering to the subject a therapeutically or prophylactically effective amount of an agent which is a nucleic acid molecule encoding a protein comprising amino acids 19 to 168 of the amino acid sequence in FIG. 2 (SEQ ID NO: 2) or a functional equivalent of this protein.

4. An agent which is a nucleic acid molecule encoding a protein comprising amino acids 19 to 168 of the amino acid sequence in FIG. 2 (SEQ ID NO: 2) or a functional equivalent of this protein for use in a method of treating or preventing a rheumatic disease in a subject.

5. The method of any one of claim 1 or 3 or the agent for use of any one of claim 2, or 4, wherein the agent is, or encodes, a protein comprising a sequence having at least 90% sequence identity to the sequence of amino acids 19 to 168 of SEQ ID NO: 2, and said protein binds C5 to prevent the cleavage of complement C5 by C5 convertase into complement C5a and complement C5b and binds to LTB4.

6. The method of any one of claim 1, 3 or 5 or the agent for use of any one of claim 2, 4 or 5, wherein the agent is, or encodes, a protein comprising a sequence having at least 95% sequence identity to the sequence of amino acids 19 to 168 of SEQ ID NO: 2, and said protein binds C5 to prevent the cleavage of complement C5 by C5 convertase into complement C5a and complement C5b and binds to LTB4.

7. The method of any one of claims 1, 3 or 5 to 6, or the agent for use of any one of claims 2, 4 or 5 to 6, wherein the agent is, or encodes, a protein comprising or consisting of the sequence of amino acids 19 to 168 of SEQ ID NO: 2.

8. The method of any one of claim 1 or 3, or the agent for use of any one of claim 2 or 4, wherein agent is, or encodes, a protein comprising the sequence of amino acids 19 to 168 of SEQ ID NO: 2, in which up to 50 amino acid substitutions, insertions or deletions have been made,
and the protein binds C5 to prevent the cleavage of complement C5 by C5 convertase into complement C5a and complement C5b and binds to LTB4,
wherein each of the six cysteine amino acids at positions 6, 38, 100, 128, 129, 150 of the mature Coversin molecule as set out in SEQ ID NO: 4 is retained and at least five, ten or fifteen or each of the LTB4 binding residues and at least five, ten or fifteen or twenty or each of C5 binding residues set is retained or is subject to a conservative modification, wherein the LTB4 binding residues are Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115 (numbering according to SEQ ID NO:4) and the C5 binding residues are Val26, Val28, Arg29, Ala44, Gly45, Gly61, Thr62, Ser97, His99, His101, Met 114, Met 116, Leu117, Asp118, Ala119, Gly120, Gly121, Leu122, Glu123, Val124, Glu125, Glu127, His146, Leu147 and Asp 149 (numbering according to SEQ ID NO:4).

9. The method or agent for use of claim 8 wherein up to 2, 3, 4, 5, 10, 15, 20 of the LTB4 and C5 binding residues are subject to a conservative modification.

10. The method or agent for use of claim 8 or 9 wherein at least five, ten or fifteen or each of the LTB4 binding residues and at least five, ten or fifteen or twenty or each of C5 binding residues is retained.

11. The method or agent for use of any of claims 8 to 10 wherein each of the LTB4 binding residues and each of C5 binding residues is retained or is subject to a conservative modification.

12. The method or agent for use of any of claims 8 to 11 wherein each of the LTB4 binding residues and each of C5 binding residues is retained or is subject to a conservative modification, wherein up to 2, 3, 4, 5, 10, 15, 20 of the C5 and/or LTB4 binding residues are subject to a conservative modification.

13. The method or agent for use of any of claims 8 to 12, wherein each of the LTB4 binding residues and each of the C5 binding residues is retained.

14. The method of any one of claim 1 or 3, or the agent for use of any one of claim 2 or 4, wherein the agent is, or encodes, a fragment of the protein as defined in any of the preceding claims,
and the protein binds C5 to prevent the cleavage of complement C5 by C5 convertase into complement C5a and complement C5b and binds to LTB4.

15. The method or the agent for use of any preceding claim, wherein the agent is administered subcutaneously or intrasynovially, preferably subcutaneously.

16. The method or the agent for use of any preceding claim, wherein the subject is a human.

17. The method or the agent for use of any preceding claim, wherein the rheumatic disease is selected from ankylosing spondylitis, relapsing polychondritis, systemic lupus erythematosus, rheumatoid arthritis, gout, inflammatory arthritis, pseudogout, juvenile arthritis, Sjögren syndrome, scleroderma, polymyositis, dermatomyositis, Behçet's disease and psoriatic arthritis.

18. The method or the agent for use of any preceding claim, wherein the rheumatic disease is RA, optionally wherein the RA is RF and/or anti-CCP positive RA.

19. The method or the agent for use of claim 18, wherein the RA is accompanied by vasculitis.

20. The method or agent for use of any preceding claim, wherein there the method comprises administering to the subject an initial ablating regimen of the agent and then administering maintenance doses of the agent, optionally wherein there is an initial maintenance dose and one or more further maintenance doses.

21. The method or the agent for use of any preceding claim, wherein the method further comprises the administration of a second rheumatic disease treatment.

22. The method or the agent for use of claim 21, wherein the second rheumatic disease treatment is selected from a DMARD, an anti-inflammatory agent (e.g. an NSAID, or a glucocorticoid) and an analgesic.

23. The method or the agent for use of claim 22 wherein:
(a) the DMARD is selected from cyclosporine, cyclophosphamide, hydroxychloroquine, gold salts, methotrexate, leflunomide, mycophenolate, sulfasalazine, etanercept, certolizumab pegol, golimumab, infliximab, and adalimumab, anakinra, rituximab and abatacept, preferably selected from etanercept, certolizumab pegol, golimumab, infliximab, and adalimumab, anakinra, rituximab and abatacept,
(b) the anti-inflammatory agent is an NSAID, or a glucocorticoid, and/or
(c) the analgesic is selected from paracetamol, compound analgesics and an opiod analgesic.

24. The method or the agent for use of any preceding claim wherein the functional equivalent of the protein comprising amino acids 19 to 168 of SEQ ID NO:2 is a fusion protein comprising (a) a sequence as defined in any of claims 6 to 14, and (b) a second sequence
and said fusion protein binds C5 to prevent the cleavage of complement C5 by C5 convertase into complement C5a and complement C5b and binds LTB4.

25. The method or agent for use of claim 24 wherein said second sequence is a PAS sequence.

26. The method or agent for use of claim 23 or 24, wherein said fusion protein comprises multiple copies of one of ASPAAPAPASPAAPAPSAPA (SEQ ID NO: 15); AAPASPAPAAPSAPAPAAPS (SEQ ID NO: 16); APSSPSPSAPSSPSPASPSS (SEQ ID NO: 17), SAPSSPSPSAPSSPSPASPS (SEQ ID NO: 18), SSPSAPSPSSPASPSPSSPA (SEQ ID NO: 19), AASPAAPSAPPAAASPAAPSAPPA (SEQ ID NO: 20) and ASAAAPAAASAAASAPSAAA (SEQ ID NO: 21), preferably 20-30 or 30 copies of one of SEQ ID NOs 15-21.

27. The method or agent for use of any of claims 24 to 26, wherein said fusion protein comprises (a) a PAS sequence consisting of 30 copies of SEQ ID NO:15 and (b) amino acids 19-168 of SEQ ID NO:2, wherein (a) is fused to the N terminus of (b).

28. The method or agent for use of any of claims 24 to 27, wherein said fusion protein comprises the sequence of SEQ ID NO:22.

29. The method or agent for use of any one of claims 1 to 28, wherein the protein or fusion protein binds C5 to prevent the cleavage of complement C5 by C5 convertase into complement C5a and complement C5b and binds LTB4.
